(19) United States
(12) Patent Application Publication
(10) Pub. No.: US 2025/0144101 A1
(43) Pub. Date: May 8, 2025

(54) LYSOSOMAL DYSFUNCTION IN NEUROLOGICAL AND PSYCHIATRIC DISORDERS

(71) Applicants: The Johns Hopkins University, Baltimore, MD (US); Sumitomo Pharma Co., Ltd., Osaka-Shi (JP)

(72) Inventors: Akira Sawa, Baltimore, MD (US); Koko Ishizuka, Baltimore, MD (US); Kun Yang, Baltimore, MD (US); Atsushi Saito, Baltimore, MD (US); Hana Kida, Tokyo (JP); Masakuni Horiguchi, Osaka (JP); Yoshio Nakai, Tokyo (JP); Tomonori Kobayashi, Tokyo (JP)

(21) Appl. No.: 18/730,684

(22) PCT Filed: Jan. 20, 2023
(86) PCT No.: PCT/US23/61044

§ 371 Date: Jul. 19, 2024

Related U.S. Application Data
(60) Provisional application No. 63/301,403, filed on Jan. 20, 2022.

(51) Int. Cl. A61K 31/519; A61K 31/22; A61K 31/351; A61K 31/40; A61K 31/427; A61K 31/4439; A61K 31/47; A61K 31/505; A61K 45/06; A61P 25/28

(52) U.S. Cl. CPC A61K 31/519; A61K 31/22; A61K 31/351; A61K 31/40; A61K 31/427; A61K 31/4439; A61K 31/47; A61K 31/505; A61K 45/06; A61P 25/28

(57) ABSTRACT

Compositions which treat lysosomal deficits in lysosomal storage disorders, neuronal ceroid lipofuscinosis, idiopathic/sporadic brain disorders that include neurological and psychiatric disorders, include agents such as troglitazone, rosuvastatin, Compound A or the combination thereof.

### CROSS-REFERENCES TO RELATED APPLICATION

This application claims the benefit of priority of U.S. Provisional Application No. 63/301,403 filed on Jan. 20, 2022, which is incorporated herein by reference in its entirety and for all purposes.

### FIELD

The disclosure relates to methods for treating a disease or disorder associated with lysosomal dysfunction that frequently accompany cognitive changes and neuropsychiatric manifestations. In particular, compositions include one or more agents which modulate lysosomal function or activities in diseases or disorders, which in turn modulate cognition and neuropsychiatric manifestations. These include, for example, Lysosomal storage disorders (LSDs), neuronal ceroid lipofuscinosis (NCLs), and idiopathic/sporadic neuropsychiatric conditions that accompany lysosomal functional deficits, such as schizophrenia (SZ) and Alzheimer's disease (AD).

### BACKGROUND

Idiopathic/sporadic, neuropsychiatric disorders have been found to display lysosomal abnormalities. Lysosomes are membrane-bound cell organelles central to degradation processes in animal cells. Extracellular materials such as microorganisms taken up by phagocytosis, macromolecules by endocytosis, and unwanted cell organelles, fuse with lysosomes and are broken down to their basic molecules. Thus, lysosomes are the recycling units of a cell. Lysosomes are also responsible for cellular homeostasis for their role in secretion, plasma membrane repair, cell signaling, and energy metabolism.

The essential role of lysosomes in cellular degradation processes puts these organelles at the crossroads of several cellular processes, with significant implications for health and disease. Defects in one of 60 lysosomal enzymes, transmembrane proteins or other components of this organelle, prevent the breakdown of target molecules, and are responsible for more than 60 different human genetic diseases, which are collectively known as lysosomal storage disorders. The large number and variety of human pathological conditions that are characterized, if not caused by aberrant lysosomal functions, underscores the critical importance of the autophagy-lysosome pathways to cellular metabolism. In these diseases as well as diseases characterized by lysosomal dysfunction, undegraded materials accumulate within the lysosomes, contributing to the presence or severity of disease ranging from lysosomal storage disorders to cancer, to cardiovascular disease. For instance, Batten disease (BD) is a group of neuropsychiatric disorders considered the most common of the neurogenetic NCLs, with a prevalence of 1 in 12,500 in some populations. There are currently no satisfactory or complete approved treatments for the various 14 forms of Batten disease.

### SUMMARY

In one aspect, we now provide evidence and the novel concept that both rare genetic diseases (e.g., BD) and idiopathic/sporadic brain disorders (e.g, SZ, AD) share common lysosomal abnormalities underlining functional deficits, in particular cognitive dysfunction.

In certain embodiments, the disease or disorder associated with lysosomal dysfunction comprises LSDs, NCLs, idiopathic/sporadic brain disorders that include a wide range of neurological, psychiatric, or functional brain disorders. In certain embodiments, the LSDs comprise glycogen storage disease, mucopolysaccaridoses, mucolipidoses, oligosaccharidoses, lipidoses, sphingolipidoses, lysosomal transport diseases, a primary lysosomal hydrolase defect, a post-translational processing defect of lysosomal enzymes, a trafficking defect for lysosomal enzymes, a defect in lysosomal enzyme protection, a defect in soluble non-enzymatic lysosomal proteins, a transmembrane (non-enzyme) protein defect or an unclassified defect, Tay-Sachs disease, Sandhoff disease, Niemann-Pick disease, Fabry disease, Krabbe disease, Farber disease, Gaucher disease, metachromatic leukodystrophy, multiple sulphatase deficiency, mucolipidosis II, mucolipidosis III, mucopolysaccharidosis, MPS III, MPS VII, GM1 gangliosidosis, and Schindler Disease. In certain embodiments, the NCLs (or BD) comprise CLN1 disease, CLN2 disease, CLN3 disease, CLN4 disease, CLN5 disease, CLN6 disease, CLN7 disease, CLN8 disease, CLN9 disease, CLN10 disease, CLN11 disease, CLN12 disease, CLN13 disease, and CLN14 disease.

In certain embodiments, the neurological diseases comprise: Alzheimer's disease, Lewy body dementia (LBD), Parkinson's Disease, prion diseases, amyotrophic lateral sclerosis (ALS), frontotemporal lobar degeneration (FTLD), Pick's disease, frontotemporal dementia with parkinsonism, multiple sclerosis, Huntington's disease, multiple system atrophy (MSA), Smith Lemli Opitz Syndrome (SLOS), Tangier disease, Pelizaeus-Merzbacher Disease, progressive supranuclear palsy, spinal muscular atrophy, and several types of spinocerebellar ataxia and other ataxic conditions. In certain embodiments, a psychiatric disorder comprises schizophrenia, mood disorders, alcoholism and other substance use disorders, autism spectrum disorder, attention deficit hyperactivity disorder, narcolepsy and other sleep disorders, a wide range of anxiety disorders.

In certain aspects, a method of treating a subject at risk of, or diagnosed with a disease or disorder associated with lysosomal dysfunction is provided, comprising: administering to the subject a therapeutically effective dose of an agent, wherein the agent preferably modulates the function or activity of a lysosome preferably within a proper homeostatic balance, thereby treating a subject at risk of, or diagnosed with a disease or disorder associated with lysosomal dysfunction. In certain embodiments, the function or activity of the lysosome within a proper homeostatic balance is determined by modulation of cellular autofluorescence (AF), lysosomal size, LAMP1 expression, lysosome enzymes or combinations thereof. In certain embodiments, the therapeutic effectiveness of the agent is monitored by cellular autofluorescence (AF), lysosomal size, LAMP1 expression, and combinations thereof.

In certain preferred aspects, the agent that modulates the function or activity of a lysosome is an organic small molecule agent. In certain preferred aspects, one or more organic small molecule agents are administered to a subject to treat a disease or disorder as disclosed herein, including one or more organic small molecule agent that preferably can modulate the function or activity of a lysosome. In one aspect, an organic small molecule agent may be considered an agent that can modulate the function or activity of a lysosome where the agent shows efficacy in vitro and/or in vivo model suitably for a specified disorder or disorder as disclosed herein, e.g. at least about a 3, 5, 10, 15, 20, 25, 30, 40, 50, 60, 70, 80 or 100 percent increase in a tested property or parameter relative to a control (e.g. a control may be the assay conducted in the absence of the organic small molecule agent). Suitably in vitro and in vivo assays are disclosed herein and/or also known.

In certain aspects, methods and uses are provided for treatment of a subject suffering from or susceptible to a neuronal ceroid lipofuscinose (NCL) disease or disorder. In certain preferred aspects, one or more organic small molecule agents are administered to a subject to treat the neuronal ceroid lipofuscinose (NCL) disease or disorder, including one or more organic small molecule agent that preferably can modulate the function or activity of a lysosome as disclosed herein.

In certain aspects, methods and uses are provided for treatment of a subject suffering from or susceptible to CLN3 disease. In certain preferred aspects, one or more organic small molecule agents are administered to a subject to treat the CLN3 disease, including one or more organic small molecule agent that preferably can modulate the function or activity of a lysosome as disclosed herein.

In certain aspects, methods and uses are provided for treatment of a subject suffering from or susceptible to Batten disease. In certain preferred aspects, one or more organic small molecule agents are administered to a subject to treat Batten disease, including one or more organic small molecule agent that preferably can modulate the function or activity of a lysosome as disclosed herein.

In certain embodiments, the one or more small molecules for administration in the present methods and uses comprise troglitazone, rosuvastatin, or the combination thereof. A set of small molecules that modulate the overall metabolism of lipids and proteins, such as troglitazone and rosuvastatin, can directly modulate (in particular, enhance) lysosomal function.

In certain embodiments, the one or more small molecules for administration in the present methods and uses may comprise one or more thiazolidinedione compounds other than or in addition to troglitazone, such as pioglitazone and/or rosiglitazone.

In certain embodiments, the one or more small molecules for administration in the present methods and uses may comprise one or more compounds that activate peroxisome proliferator-activated receptors (PPARs). In certain embodiments, the one or more small molecules for administration in the present methods and uses may comprise one or more compounds that activate PPAR-γ.

In further embodiments, the one or more small molecules for administration in the present methods and uses may comprise one or more statin compounds. In particular, the one or small molecule compound may comprise one or more of atorvastatin (Lipitor), fluvastatin (Lescol XL), lovastatin (Altoprev), pitavastatin (Livalo), pravastatin (Pravachol), rosuvastatin (Crestor, Ezallor) and/or simvastatin (Zocor, FloLipid).

In certain embodiments, the agent comprises other small molecule compounds, antisense oligonucleotides, siRNA reagents, antibodies, antibody fragments, single chain antibodies, antibody mimetics, peptoids, aptamers; enzymes, peptides, organic or inorganic molecules, natural or synthetic compounds or combinations thereof. In certain embodiments, the agent comprises one or more small molecule compounds. In certain embodiments, the one or more small molecules modulate glycolipid metabolism. In certain embodiments, the method further comprises administering a secondary therapeutic agent.

In another aspect, a method of identifying a candidate therapeutic agent (including an organic small molecule agent) comprises contacting a cell with a candidate therapeutic agent, wherein the cell comprises a lysosomal deficit; assaying for modulation of function of the lysosome as compared to a normal control; thereby identifying a candidate therapeutic agent. In certain embodiments, the candidate therapeutic agent modulates cellular autofluorescence (AF), lysosomal size, LAMP1 expression, lysosome enzymes and combinations thereof to maintain their homeostatic balance.

In certain embodiments, the candidate therapeutic agent comprises small molecule compounds, antisense oligonucleotides, siRNA reagents, antibodies, antibody fragments, single chain antibodies, antibody mimetics, peptoids, aptamers; enzymes, peptides, organic or inorganic molecules, natural or synthetic compounds or combinations thereof. In certain embodiments, the cell comprises: stem cells, cord blood cells, adult stem cells, mesenchymal stem cells, mesenchymal stromal cells, induced pluripotent stem cells (iPSC), autologous cells, autologous stem cells, bone marrow cells, hematopoietic cells, hematopoietic stem cells, somatic cells, germ line cells, differentiated cells, somatic stem cells, embryonic stem cells, cell lines, patient cells, mammalian cells, peripheral blood mononuclear cells (PBMCs), leukocyte, neural cells, brain cells or central nervous system cells. In certain embodiments, the cell comprises comprising: autologous cells, allogeneic cells, haplotype matched cells, haplotype mismatched cells, haplo-identical cells or xenogeneic cells.

In an aspect, the small molecule agent (including in use in a method of treating a subject at risk of, or diagnosed with a disease or disorder disclosed herein) is a compound having a structure of:

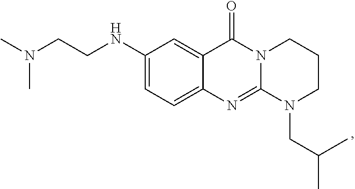 [SMILES: CC(C)CN1CCCn2c1nc1ccc(NCCN(C)C)cc1c2=O]

or a pharmaceutically acceptable salt thereof.

In certain embodiments, the disease or disorder associated with lysosomal dysfunction comprises lysosomal storage disorders (LSDs), neuronal ceroid lipofuscinoses (NCLs), idiopathic/sporadic brain disorders that include neurological, psychiatric, or functional brain disorders, and in particular, the disease is Batten Disease (BD). In certain embodiments, the NCLs or BD comprise CLN3 disease. In certain embodiments, the compound can control or modulate expression of PLA2 gene, particularly, PLA2G4A and/or PLA2G7 genes. In certain aspects, the compound may influence the disease pathology occurring in hippocampal CA3 region or cerebral cortex in brain.

### Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used in this specification and the appended claims, the term “or” is generally employed in its sense including “and/or” unless the content clearly dictates otherwise. Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The terms “administer,” “administration,” or “administering” as used herein refer to (1) providing, giving, dosing and/or prescribing by either a health practitioner or his authorized agent or under his direction a composition according to the disclosure, and (2) putting into, taking or consuming by the patient or person himself or herself, a composition according to the disclosure.

As used herein, the term “agent” or “candidate therapeutic agent” is meant to encompass any molecule, chemical entity, composition, drug, therapeutic agent, chemotherapeutic agent, or biological agent capable of preventing, ameliorating, or treating a lysosomal dysfunction or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. Agents include compounds that are known drugs, compounds for which therapeutic activity has been identified but which are undergoing further therapeutic evaluation, and compounds that are members of collections and libraries that are to be screened for a pharmacological activity. An agent can be assayed in accordance with the methods of the invention at any stage during clinical trials, during pre-trial testing, or following FDA-approval.

As used herein “cognitive function” can mean any mental process that involves perception, variety types of memory including working memory, emotional regulation, awareness, attention, reasoning, thinking and capacity for judgment, flexibility of thinking, and executive functions. Measures of cognitive functioning include assessment tools designed to measure, for example: (a) general intelligence, (b) nonverbal intelligence, (c) achievement, (d) attention/executive functioning, (e) memory, (f) visual-motor and motor functioning, (g) emotional and anxiety regulation, (h) reward process, motivation, and hedonic measures, (i) cognitive flexibility, set shifting, and reversal learning, and (j) language. Such assessment tools are well-known in the art and include, for example, Wechsler Adult Intelligence Scale and Woodcock-Johnson III Tests of Cognitive Abilities (both for assessing general intelligence), Raven Progressive Matrices (for assessing nonverbal intelligence), Wide Range Achievement Test and Woodcock-Johnson III Tests of Achievement (for assessing academic achievement), Conners' Continuous Performance Test II (for assessing attention/executive functioning), Wide Range Assessment of Memory and Learning (for assessing memory and learning), Bender Visual-Motor Gestalt Test, Halstead-Reitan Grip Strength Test, Halstead-Reitan Finger Tapping Test and Lafayette Grooved Pegboard Task (all for assessing visual-motor and motor functioning), the Cambridge Neuropsychological Test Automated Battery (CANTAB) Emotion Recognition Task, The Penn Computerized Neurocognitive Battery (PCNB) Penn Emotion Identification Test and Penn Emotion Differentiation Test, Mayer-Salovey-Caruso Emotional Intelligence Test (MSCEIT™): Managing Emotions, Emotion Amplification and Reduction Scales, Emotion Regulation Questionnaire, Zung Self-Rating Anxiety Scale, Hamilton Anxiety Scale, Beck Anxiety Inventory, Penn State Worry Questionnaire, Intolerance of Uncertainty Scale, Behavioral Inhibition Scale, Fear of Negative Evaluation Scale, Anxiety Sensitivity Index, and Life Events and Difficulties Schedule (for assessing emotional and anxiety regulation), Reinforcement learning, Monetary incentive reward, Adapted Cambridge gambling, Progressive ratio, Simple guessing task, Fixed-ratio Satiation Schedule, Devaluation Task, Habit Task, Habit Learning Task, Probabilistic Reward Task, Pavlovian Conditioning, Drifting Double Bandit, Probability Choice Task, Willingness To Pay Task, Delayed Discounting Task, and Effort Expenditure for Reward Task (for assessing reward process, motivation, and hedonic measures), CANTAB Intra-Extra Dimensional Set Shift, PCNB Penn Conditional Exclusion Test, and Wisconsin Card Sorting Test (for assessing cognitive flexibility, set shifting, and reversal learning) and Peabody Picture Vocabulary Test (for assessing language). The phrase “improving cognitive function”, as referred to herein, means a positive change in the ability of the subject to perform a symbolic operation, for example, to perceive, remember, create a mental image, have clarity of thought, be aware, to reason, think or judge. The term “decrease in cognitive function” as used herein refers to a negative change in the ability of the subject to perform a symbolic operation, for example, to perceive, remember, create a mental image, have clarity of thought, be aware, to reason, think or judge.

The term “combination therapy”, as used herein, refers to those situations in which two or more different agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “contacting” has its normal meaning and refers to combining two or more agents, combining agents and cells, or combining two populations of different cells. Contacting can occur in vitro, e.g., mixing troglitazone and/or rosuvastatin and a cell or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject or a non-human animal, e.g., by administering an agent to a subject for delivery the agent to a target cell.

The terms “determining”, “measuring”, “evaluating”, “detecting”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

As used herein, “diagnosis” means identifying the presence, extent and/or nature of a pathologic condition. Diagnostic methods differ in their specificity and selectivity. While a particular diagnostic method may not provide a definitive diagnosis of a condition, it suffices if the method provides a positive indication that aids in diagnosis.

As used herein, “lysosomal dysfunction” or “lysosomal deficit” is meant any activity, enzymatic or non-enzymatic, or any property of the lysosomes that is affected in a negative manner relative to a control. This includes vesicle trafficking to or from lysosomes, the endocytic pathway, heterophagy or autophagy, and including the expression and activity of enzymes that are localized in the lysosomes. By “modulating a lysosomal function” is meant increasing or decreasing one or more such activities from the level or amount of such activity found in a normal control state, or within the proper homeostatic balance. Examples of lysosomal enzymes that can be imodulated include a lysosomal acid hydrolase, lysosomal protease, lysosomal nuclease, lysosomal lipase, amylase and a cathepsin. cathepsin B, cathepsin H or cathepsin L can be assayed using methods known in the art, for example, as described by Barrett, A. J. et al., Meth. Enzymol. 80:535 (1981), Academic Press, New York, incorporated herein by reference. Lysosomal dysfunction or deficit is further described as an abnormal lysosomal morphology, chemistry or activity, which is detrimental to lysosomes or cells. Examples of lysosomal dysfunctions include a detrimental change, either increased or decreased, in the normal activity of the endocytic pathway, a detrimental change in lysosomal morphology, a detrimental change in the intra-lysosomal pH, and/or the activity(ies) of lysosomal enzyme(s).

The term “modulator” is used to refer to an entity whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

A “pharmaceutically acceptable salt” as referred to herein, is any salt preparation that is appropriate for use in a pharmaceutical application. Pharmaceutically acceptable salts include, but are not limited to, amine salts, such as N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane and the like; alkali metal salts, such as lithium, potassium, sodium and the like; alkali earth metal salts, such as barium, calcium, magnesium and the like; transition metal salts, such as zinc, aluminum and the like; other metal salts, such as sodium hydrogen phosphate, disodium phosphate and the like; mineral acids, such as hydrochlorides, sulfates and the like; and salts of organic acids, such as acetates, lactates, malates, tartrates, citrates, ascorbates, succinates, butyrates, valerates, fumarates and the like.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

“Pharmaceutical composition” refers to a composition suitable for pharmaceutical use in a subject animal, including humans and mammals. A pharmaceutical composition comprises a therapeutically effective amount of a treatment agent as disclosed herein (e.g., an agent modulates the function or activity of a lysosome, which in turn modulates cognitive function) optionally another biologically active agent, and optionally a pharmaceutically acceptable excipient, carrier or diluent. In an embodiment, a pharmaceutical composition encompasses a composition comprising the active ingredient(s), and the inert ingredient(s) that make up the carrier, as well as any product that results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the disclosure and a pharmaceutically acceptable excipient, carrier or diluent.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “prophylactic” treatment is a treatment administered to a subject who does not exhibit signs of a disease or exhibits only early signs of the disease, for the purpose of decreasing the risk of developing pathology. The compounds or compositions of the disclosure may be given as a prophylactic treatment to reduce the likelihood of developing a pathology or to minimize the severity of the pathology, if developed.

As used herein, the term “subject” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender. In various embodiments the subject is human.

A “therapeutic” treatment is a treatment administered to a subject who exhibits signs or symptoms of pathology for the purpose of diminishing or eliminating those signs or symptoms. The signs or symptoms may be biochemical, cellular, histological, functional or physical, subjective or objective.

“Treatment” refers to prophylactic treatment or therapeutic treatment. In certain embodiments, “treatment” refers to administration of a compound or composition to a subject for therapeutic or prophylactic purposes.

As used herein, the term “unit dosage form” refers to physically discrete units suitable as unitary dosages for human and animal subjects, each unit containing a predetermined quantity of a compound of the disclosure calculated in an amount sufficient to produce the desired effect, optionally in association with a pharmaceutically acceptable excipient, diluent, carrier or vehicle. The specifications for the novel unit dosage forms of the present disclosure depend on the particular compound employed and the effect to be achieved, and the pharmacodynamics associated with each compound in the host.

References herein to “Compound A” means a compound of the following structure:

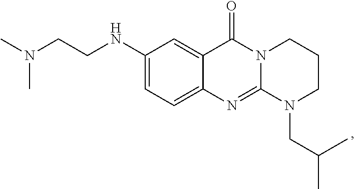 [SMILES: CC(C)CN1CCCn2c1nc1ccc(NCCN(C)C)cc1c2=O]

or a pharmaceutically acceptable salt thereof.

### BRIEF DESCRIPTION OF THE DRAWINGS

FIGS. 1A-1C are a series of plots and an electron microscopic image demonstrating that several types of lysosome-associated abnormalities found in cells from patients with schizophrenia (SZ), correlate with cognitive changes. FIG. 1A: Cellular autofluorescence (AF) were significantly elevated in peripheral blood cells from schizophrenia (SZ) patients, compared with those from healthy controls (CON). FIG. 1B: Altered lysosome was detected in peripheral blood cells from some of SZ patients by immune-electron microscopy. We detected the alternation by shape and size of the organelle and accumulation of GAPDH in lysosome, a sign of stress signaling. Green triangles indicate the boundary of lysosomes; red triangles indicate GAPDH immunoreactivity. FIG. 1C: Levels of AF and cognitive flexibility (Wisconsin Card Sorting Test) scores were negatively correlated. CON (white); SZ (black).

FIGS. 2A-2D are a series of plots and graphs showing results from the BD cell model. FIG. 2A: Correlation between intensity of autofluoresence (AF) and area of LAMP1 in lymphoblasts from healthy (white circles) and Juvenile NCL (JNCL) patient (green circles). FIG. 2B: Lymphoblasts form JNCL patient showed elevated AF. Troglitazone (3 μM) and rosuvastatin (10 μM) decreased it. FIG. 2C: Lymphoblasts form JNCL patient heavily stained with anti-LAMP1 antibody. Troglitazone (3 μM) and rosuvastatin (10 μM) decreased it (n=3). FIG. 2D: Neuronal cells differentiated from Cln3 mutant iPSCs are heavily stained with anti-LAMP1 antibody. Troglitazone (0.08-2 μM) and rosuvastatin treatment (0.4-10 μM) dose-dependently decreased it (n=2-3).

FIGS. 3A-3C are a series of immunostains and graphs showing results from the BD animal model (Cln3^{-/-} mice). FIG. 3A: Representative figures of immunohistochemistry of LAMP1 of the brain sections (CA2/3 and VPL/VPM) from 6-month old wild-type mice, untreated and troglitazone (0.06-0.3 mg/g food, 4 months)-treated Cln3^{-/-} mice (left). Quantitative analysis of LAMP1 positive area in CA2/3 and VPL/VPM. We found the accumulation of LAMP1 in untreated Cln3^{-/-} mice brains, whereas brains from troglitazone-treated mice showed reduction of LAMP1 positive area (right, n=8-10). FIG. 3B: Novel object recognition (NOR) test with 6-month old wild-type mice, untreated and troglitazone (0.06-3 mg/g food, 4 months)-treated Cln3−/− mice. Untreated Cln3−/− mice showed the impairment in NOR test, whereas troglitazone-treated Cln3−/− mice did not. FIG. 3C: Peripheral white blood cells from Cln3−/− mice heavily stained with anti-LAMP1 antibody. Treatment with troglitazone (0.06-3 mg/g food, 4 months) decreased it (n=8-10).

FIGS. 4A to 4D show beneficial effects of Compounds A on Batten-associated pathology in human cells, and mouse disease model (cell pathology, behavior). A, amelioration of elevated autofluorescence (AF) in Batten disease (BD) lymphoblasts by Compound A. B, amelioration of elevated LAMP1 signal in Batten disease (BD) lymphoblasts by Compound A. C, amelioration of elevated LAMP1 signal in peripheral white blood cells of BD mice [CLN3 knockout (KO) mice] by Compound A. D, Amelioration of cognitive deficits (novel object recognition test) of BD mice by Compound A.

FIGS. 5A and 5B show expression control of PLA2G4A (FIG. 5A) and PLA2G7 (FIG. 5B) by Compound A. Compound A significantly influenced the expression of PLA2 genes, which may underlie a main mechanism of pathological amelioration of Batten CLN3 disease. The data was obtained through RNA-sequencing analysis in which we compared gene expression among the groups (i) wild-type (WT) neurons+mock, (ii) WT neurons+Compound A, (iii) Batten cell model CLN3 knockout (KO) neurons+mock, and (iv) KO neurons+Compound A. Neurons were prepared by differentiating human induced pluripotent stem cells.

FIGS. 6A and 6B show connection between the cellular/tissue pathology with the behavioral/neuropsychiatric phenotypes by conducting the histological analysis of autofluorescence (AF). FIG. 6A shows that the Batten CLN3 disease pathology in the brain was emphasized in the hippocampal CA3 region, and FIG. 6B also shows that the cerebral cortex was involved in CLN3 KO mice. Thus, the influence of Compound A on PLA2 genes may be a main mechanism of pathological amelioration, partly because PLA2 genes are directory involved in the metabolism of G3PI.

### DETAILED DESCRIPTION

Idiopathic/sporadic brain disorders have been found to display lysosomal abnormalities. These deficits are underscored in, for example, Alzheimer's disease (AD). Lysosomal deficit is likely to involved in pathophysiology of psychiatric disorders. For example, alternation in gene expression profiles in SZ postmortem brains from patients with schizophrenia (SZ) has been reported. However, the investigation of lysosomal deficit in psychiatric disorders is still in its beginnings. Without wishing to be bound by theory, it was hypothesized that lysosomal deficit can be a primary driver for neuronal dysfunction.

There are a group of rare genetic disorders in which lysosomal dysfunction is regarded as its central pathology. One of the representative disorders is BD, which is also called NCLs. BD is can be characterized with vision problems, epilepsy, cognitive deficit including emotional/anxiety regulation and psychosis. Patients with BD start to become dysfunctional in their childhood, and can only survive for 20-30 years. Mutations in the gene called Cln3 cause about half cases of BD, and there are several other outstanding genes such as Cln2. Cln3-driven BD is regarded as a homogeneous condition that represents the lysosomal pathology. Accordingly, animal and cell models that carry disease-associated mutation(s) of Cln3 (or loss-of-function of Cln3) have been frequently used in research, since the gene was identified in 1995. These models are indeed regarded as effective to explore lysosomal deficits in brain disorders.

### Compositions

In one aspect, the present disclosure provides a composition comprising an agent that modulates glycolipid metabolism as the active ingredient. In certain embodiments, a composition comprises a therapeutically effective dose of at least one agent, wherein the agent modulates the function or activity of the lysosome. In certain embodiments, the function or activity of the lysosome is determined by modulation of cellular autofluorescence (AF), lysosomal size, LAMP1 expression, lysosome enzymes or combinations thereof. In certain embodiments, the therapeutic effectiveness of the agent is monitored by cellular autofluorescence (AF), lysosomal size, LAMP1 expression, and combinations thereof.

In certain embodiments, a composition comprises a pharmaceutical composition comprising troglitazone and rosuvastatin, a set of small molecules that modulate the overall metabolism of lipids and proteins, which in turn, can directly modulate (in particular, enhance) lysosomal function.

In certain embodiments, a composition comprises a pharmaceutical composition comprising other small molecule compounds, antisense oligonucleotides, siRNA reagents, antibodies, antibody fragments, single chain antibodies, antibody mimetics, peptoids, aptamers; enzymes, peptides, organic or inorganic molecules, natural or synthetic compounds or combinations thereof.

In certain embodiments, a composition comprises a pharmaceutical composition comprising one or more small molecule compounds. In certain embodiments, the one or more small molecules modulate glycolipid metabolism. In certain embodiments, the one or more small molecules comprise troglitazone, rosuvastatin or the combination thereof.

As discussed, in certain embodiments, the one or more small molecules may comprise one or more thiazolidinedione compounds other than or in addition to troglitazone, such as pioglitazone and/or rosiglitazone. In certain embodiments, the one or more small molecules may comprise one or more compounds that activate peroxisome proliferator-activated receptors (PPARs). Suitable troglitazone compounds are also disclosed in U.S. Pat. No. 6,207,690.

In certain embodiments, the one or more small molecules may comprise a compound having a structure of:

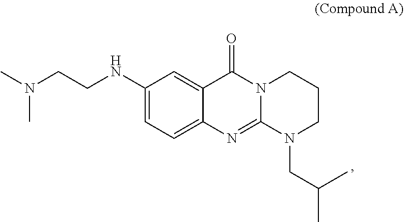 [SMILES: CC(C)CN1CCCn2c1nc1ccc(NCCN(C)C)cc1c2=O]

or a pharmaceutically acceptable salt thereof.

Compound A may be readily prepared. The following Scheme 1 is exemplary:

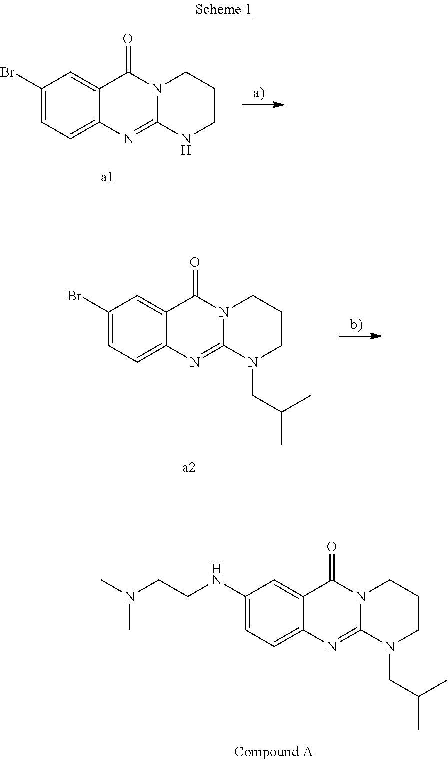 [SMILES: O=c1c2cc(Br)ccc2nc2n1CCCN2 ; CC(C)CN1CCCn2c1nc1ccc(Br)cc1c2=O ; CC(C)CN1CCCn2c1nc1ccc(NCCN(C)C)cc1c2=O]

As shown in the Scheme 1 in step a) suitably aryl halide a1 may be reacted under basic conditions to substitute the ring nitrogen with the alkyl group. For instance, an electrophilic reagent such as an alkyl halide may be reacted to provide the substituted ring nitrogen compound exemplified by a2 in the above Scheme 1. Reagents such as a1 may be commercially available or readily prepared such as by methods described in Tetrahedron (2007), 63(4), 847-854. As shown in step b) in the above Scheme 1, compound a2 is further reacted to provide desired substitution as depicted for Compound A.

In further embodiments, the one or more small molecules may comprise one or more statin compounds. In particular, the one or small molecule compound may comprise one or more of atorvastatin, fluvastatin, lovastatin, pitavastatin, pravastatin, rosuvastatin and/or simvastatin.

In certain embodiments, a composition comprises a pharmaceutical composition comprising at least one of Kaempferide (3,5,7-trihydroxy-4′-methoxyflavone), imiglucerase, velaglucerase, taliglucerase, miglustat, eliglustat, agalsidase beta, agalsidase alpha, migalastat, laronidase, idursulfase, sulphamidase, elosulphase, galsulphase, sebelipase, cerliponase or combinations thereof.

In certain embodiments, a composition comprises a pharmaceutical composition comprising at least one substance which modulates phospholipase A2 (PLA2) expression or activity.

In certain embodiments, PLA2 includes secreted PLA2 (group I, II, III, V, IX, X, XI, XII, XIII and XIV; e.g. PLA2G1A, PLA2G1B, PLA2G2A, PLA2G2B, PLA2G2C, PLA2G2D, PLA2G2E, PLA2G2F, PLA2G3, PLA2G5, PLA2G9, PLA2G10, PLA2G11A, PLA2G111B, PLA2G12A, PLA2G12B, PLA2G13 and PLA2G14), cytosolic PLA2 (group IV; e.g. PLA2G4A, PLA2G4B, PLA2G4C, PLA2G4D, PLA2G4E and PLA2G4F), Ca²-independent PLA2 (group VI; e.g. PLA2G6A, PLA2G6B, PLA2G6C, PLA2G6D, PLA2G6E and PLA2G6F), platelet-activating factor-acetyl hydrolases (group VII and VIII; e.g. PLA2G7A, PLA2G7B, PLA2G8A and PLA2G8B), lipoprotein-associated PLA2 (group XV; e.g. PLA2G15), adipose-PLA2 (group XVI; e.g. PLA2G16) (Dennis et al., Chem Rev. 111: 6130 (2011)), or combinations thereof.

TABLE 1
Overview of approved drugs for treating lysosomal storage disorders and examples
of some products under development with an orphan drug designation.

| | | |
|---|---|---|
| Gaucher disease | Imiglucerase (Cerezyme, enzyme | Approved in Europe, |
| | preparation, produced in CHO | USA, and other countries |
| | cells) | |
| | Velaglucerase (Vpriv, enzyme | Approved in Europe, USA |
| | preparation, produced in human | and other countries |
| | cells) | |
| | Taliglucerase (Elelyso, enzyme | Approved in USA, Brazil |
| | preparation, produced in plant | and Canada |
| | cells) | |
| | Miglustat (Zavesca, substrate | Approved in Europe |
| | reduction) | Approved in Europe, |
| | Eliglustat (Cerdelga, substrate | USA, and other countries |
| | reduction) | |
| Fabry disease | Agalsidase beta (Fabrazyme, | Approved in Europe, |
| | enzyme preparation, produced in | USA, and other countries |
| | CHO cells) | |
| | Agalsidase alfa (Replagal, enzyme | Approved in Europe, |
| | preparation, produced in human | Brazil, and Canada |
| | cells) | |
| | Migalastat (Galafold, Chaperone) | Approved in Europe for |
| | | patients with amenable |
| | | mutations |
| MPS I | Laronidase (Aldulrazyme, enzyme | Approved in Europe, |
| (Hurler, Hurler- | preparation, produced in CHO | USA, and other countries |
| Scheie, Scheie) | cells) | |
| | HIRMAb fusion protein | Orphan drug designation |
| MPS II (Hunter | Idursulfase (Elaprase, enzyme | Approved in Europe, |
| syndrome) | preparation, produced in human | USA, and other countries |
| | cells, for intravenous application) | |
| | Idursulfase for intrathecal | Orphan drug designation |
| | application) | |
| | HIRMAb fusion protein | Orphan drug designation |
| MPS IIIA | Sulfamidase for intrathecal | Orphan drug designation |
| (Sanfilippo A) | application | |
| | Gene therapy | Orphan drug designation |
| MPS IIIB | Insulin growth factor II fusion | Orphan drug designation |
| (Sanfilippo B) | protein for intravenous application | |
| | Gene therapy | Orphan drug designation |
| MPS IVA | Elosulfase (Vimizim, enzyme | Approved in Europe, |
| (Morquio A) | preparation, produced in CHO | USA, and other countries |
| | cells) | |
| MPS VI | Galsulfase (Naglazyme, enzyme | Approved in Europe, |
| (Maroteaux- | preparation, produced in CHO | USA, and other countries |
| Lamy) | cells) | |
| MPS VII | Recombinant enzyme preparation | Orphan drug designation |
| Pompe disease | Alglucosidas alfa (Myozyme, | Approved in Europe and |
| | enzyme preparation, produced in | other countries |
| | CHO cells) | Approved in USA |
| | Alglucosidas alfa (Lumizyme, | |
| | enzyme preparation, produced in | |
| | CHO cells) | |
| | Enzyme plus chaperone | Orphan drug designation |
| Metachromatic | Enzyme preparation, intrathecal | Orphan drug designation |
| leukodystrophy | application | |
| | Gene therapy | Orphan drug designation |
| Lysosomal acid | Sebelipase (Kanuma, enzyme | Approved in Europe and |
| lipase deficiency | preparation, produced in egg white | USA |
| (Wolman | from genetically modified chicken) | |
| disease/cholesteryl | | |
| ester storage | | |
| disease) | | |
| Neuronal ceroid | Cerliponase (Brineura, enzyme | Approved in USA, |
| lipofuscinosis type | preparation for intrathecal | positive opinion for |
| 2 | application) | marketing authorization in |
| | | Europe |
| Niemann-Pick | Recombinant enzyme | Orphan drug designation |
| disease type B | | |
| Niemann-Pick | Miglustat (Zavesca, substrate | Approved in Europe |
| disease type C | reduction) | |
| | Heat shock protein 70 | Orphan drug designation |
| α-Mannosidosis | Recombinant enzyme | Orphan drug designation |
| Farber disease | Recombinant enzyme | Orphan drug designation |

CHO, Chinese hamster ovary cells; MPS, mucopolysaccharidosis; HIRMAb, human insulin receptor monoclonal antibody.

### Methods of Treatment and Combination Therapies

In certain embodiments, a method of treating a subject at risk of, or diagnosed with a disease or disorder associated with lysosomal dysfunction, comprises administering to the subject a therapeutically effective dose of an agent (including an organic small molecule agent), wherein the agent modulates the function of the lysosome, thereby treating a subject at risk of, or diagnosed with a disease or disorder associated with lysosomal dysfunction.

In certain embodiments, the subject is administered a composition comprising a therapeutically effective dose of at least one agent (including an organic small molecule agent), wherein the agent modulates the function or activity of the lysosome. In certain embodiments, the function or activity of the lysosome is determined by modulation of cellular autofluorescence (AF), lysosomal size, LAMP1 expression, lysosome enzymes or combinations thereof. In certain embodiments, the therapeutic effectiveness of the agent is monitored by cellular autofluorescence (AF), lysosomal size, LAMP1 expression, and combinations thereof.

In certain embodiments, the subject is administered a composition comprising a pharmaceutical composition comprising troglitazone and rosuvastatin, a set of small molecules that modulate the overall metabolism of lipids and proteins, which in turn, can directly modulate (in particular, enhance) lysosomal function.

In certain embodiments, the subject is administered a composition comprising a pharmaceutical composition comprising other small molecule compounds, antisense oligonucleotides, siRNA reagents, antibodies, antibody fragments, single chain antibodies, antibody mimetics, peptoids, aptamers; enzymes, peptides, organic or inorganic molecules, natural or synthetic compounds or combinations thereof.

In certain embodiments, the subject is administered a composition comprising a pharmaceutical composition comprising at least one of Kaempferide (3,5,7-trihydroxy-4′-methoxyflavone), imiglucerase, velaglucerase, taliglucerase, miglustat, eliglustat, agalsidase beta, agalsidase alpha, migalastat, laronidase, idursulfase, sulphamidase, elosulphase, galsulphase, sebelipase, cerliponase or combinations thereof.

In certain embodiments, the subject is administered a composition comprising a pharmaceutical composition comprising one or more thiazolidinedione compounds.

In certain embodiments, the subject is administered a composition comprising a pharmaceutical composition comprising one or more thiazolidinedione compounds other than or in addition to troglitazone, such as pioglitazone and/or rosiglitazone.

In certain embodiments, the subject is administered a composition comprising a pharmaceutical composition comprising one or small molecules for administration in the present methods and uses may comprise one or more compounds that activate peroxisome proliferator-activated receptors (PPARs). In certain embodiments, the one or more small molecules for administration in the present methods and uses may comprise one or more compounds that activate PPAR-γ.

In certain embodiments, the subject is administered a composition comprising a pharmaceutical composition comprising one or more statin compounds. In particular, the one or small molecule compound may comprise one or more of atorvastatin (Lipitor), fluvastatin (Lescol XL), lovastatin (Altoprev), pitavastatin (Livalo), pravastatin (Pravachol), rosuvastatin (Crestor, Ezallor) and/or simvastatin (Zocor, FloLipid).

In certain embodiments, the subject is administered a composition comprising a pharmaceutical composition comprising Compound A.

In certain embodiments, the subject is administered a composition comprising a pharmaceutical composition comprising one or more small molecule compounds. In certain embodiments, the one or more small molecules modulate glycolipid metabolism. In certain embodiments, the one or more small molecules comprise troglitazone, rosuvastatin or the combination thereof.

In certain embodiments, the one or more therapeutic agents, e.g. troglitazone, rosuvastatin or the combination thereof, or a statin compound or Compound A, are combined with one or more secondary therapies or therapeutic agents. These include hematopoietic stem cell transplantation (HSCT), enzyme replacement therapy (ERT), pharmacological chaperone therapy (PCT), substrate reduction therapy (SRT) and gene therapy (GT) (Parenti G, et al. New strategies for the treatment of lysosomal storage diseases (Review). Int J Mol Med 31: 11-20, 2013. Michael Beck, Treatment strategies for lysosomal storage disorders. Developmental Medicine & Child Neurology 2018, 60: 13-18).

Small molecule agents for use in the present therapeutic methods may be identified and chemically synthesized using known methodology. Small molecules are usually less than about 2000 Daltons in size or alternatively less than about 1500, 750, 500, 250 or 200 Daltons in size, wherein such small molecules that are capable of providing a result in vitro or in vivo (including in vivo models) as disclosed herein, In this regard, it is noted that techniques for screening small molecule libraries for molecules that are capable of binding to a polypeptide target are well known in the art (see, e.g., PCT Publication Nos. WO00/00823 and WO00/39585). Small molecules may be, for example, fused ring systems (including those that contain 2, 3 or more fused rings, and one or more N, O or S ring atoms such as (Compound A disclosed herein) and contain one or more other functional groups such as amines (primary or more preferably secondary or tertiary alkylamine moieties), aldehydes, ketones, epoxides, or alcohols.

The small molecule chemical compound may be a component of a combinatorial chemical library. Combinatorial chemical libraries are a collection of multiple species of chemical compounds comprised of smaller subunits or monomers. Combinatorial libraries come in a variety of sizes, ranging from a few hundred to many hundreds of thousand different species of chemical compounds. There are also a variety of library types, including oligomeric and polymeric libraries comprised of compounds such as carbohydrates, oligonucleotides, and small organic molecules, etc. Such libraries have a variety of uses, such as immobilization and chromatographic separation of chemical compounds, as well as uses for identifying and characterizing ligands capable of binding an acceptor molecule or mediating a biological activity of interest. Various techniques for synthesizing libraries of compounds on solid-phase supports are known in the art. Solid-phase supports are typically polymeric objects with surfaces that are functionalized to bind with subunits or monomers to form the compounds of the library. Synthesis of one library typically involves a large number of solid-phase supports. To make a combinatorial library, solid-phase supports are reacted with one or more subunits of the compounds and with one or more numbers of reagents in a carefully controlled, predetermined sequence of chemical reactions. That is, the library subunits are “grown” on the solid-phase supports. The larger the library, the greater the number of reactions required, complicating the task of keeping track of the chemical composition of the multiple species of compounds that make up the library. In some embodiments, the small molecules are less than about 2000 Daltons in size, alternatively less than about 1500, 750, 500, 250 or 200 Daltons in size.

Hematopoietic Stem Cell Transplantation: HSCT relies on the use of hematopoietic stem cells derived from a healthy donor as a therapeutic agent. By this approach, a dual effect may be achieved; the first is to repopulate specific tissues by the donor's healthy cells, and the second and key effect is related to the secretion of functional lysosomal hydrolases by the donor's cells in the extracellular space and into the blood circulation. The secreted normal enzyme may be taken up by the recipient cells and may cross-correct the enzyme defect in these cells. A significant advantage of HSCT is that, as donor-derived, enzyme-producing cells are able to migrate to the brain. This procedure has the potential to improve neurocognitive function and quality of life, particularly when performed early in the course of the disease.

Enzyme Replacement Therapy: ERT is based on periodic intravenous infusions of human recombinant lysosomal enzymes produced and purified on large scale from different sources with recombinant DNA techniques. Once injected, the recombinant wild-type enzymes are distributed to tissues, internalized by cells and targeted to the lysosomal compartment, where they replace the defective enzyme.

Small Molecule Pharmacological Chaperones: Pharmacological chaperone therapy is based on the concept that loss-of-function diseases are often due to missense mutations that cause misfolding (abnormal conformation) of mutant proteins. The misfolded proteins are recognized by the quality control systems of the endoplasmic reticulum and are degraded. Thus, in these so-called ‘misfolding protein diseases’ the loss of function is not due to the loss of catalytic activity, but is the result of the degradation of the aberrant protein. It has been shown that small-molecule ligands (pharmacological chaperones) can interact with the mutant protein, favor its correct conformation, and enhance its stability. As a result, the enzymatic activity of the mutant protein is partially rescued. The use of pharmacological chaperones for the treatment of lysosomal storage diseases was first proposed for Fabry disease. In cells from patients with Fabry disease, one of the most potent inhibitors of α-gal A, 1-deoxygalactonojirimycin (DGJ), was paradoxically shown to enhance residual α-galactosidase A activity. In principle, chaperones (as with substrate reducing drugs and small molecule drugs in general) have several advantages, as compared to ERT, as they can be administered orally, allowing for a non-invasive treatment, are non-immunogenic and do not need to be delivered to cells through the mannose-6-phosphate pathway, which may be secondarily impaired in some of these disorders. In addition, small molecule chaperones are expected to diffuse freely across cell membranes and reach therapeutic concentrations in different tissues and systems, including the CNS. In the animal model of GM1 gangliosidosis, the use of the chaperone N-octyl 4-epi-β-valienamine (NOEV) resulted in increased residual activity in the brain and greater clearance of substrate.

Proteostasis regulators: While chaperones are ligands that specifically interact with mutant proteins (and are thus able to rescue a single protein), other drugs are able to adjust the cellular mechanisms controlling the homeostasis of proteins, or proteostasis. Proteostasis is a complex network that controls protein synthesis, folding, trafficking, aggregation and degradation. These small molecule drugs, known as proteostasis regulators, can adjust the capacity of this network and have the potential to restore the normal balance between protein folding, trafficking, and degradation. Two proteostasis regulators have been reported to restore the function of two mutant lysosomal enzymes in two distinct LSDs, Gaucher disease and GM2 gangliosidosis. Co-administration of a pharmacological chaperone and a proteostasis regulator exhibited synergy and resulted in further enhancement of enzyme activity. This approach remains largely experimental.

Gene therapy: Gene therapy has the greatest potential for complete and sustained correction of the enzymatic defect. Gene therapy is aimed at increasing or restoring the activity of the defective enzyme in the patient's cells. This is not obtained by supplying the missing enzymatic protein, but by delivering the normal copy of the defective gene, that will direct the synthesis of the normal enzyme by the recipient's cells. Different viral vectors and different strategies have been explored to accomplish in vivo gene transfer. Herpesviruses, lentiviruses, adeno-associated viruses (AAVs), adenoviruses (Ads) and others have been tested as vectors. Genetic modification may be performed either ex vivo or in vivo. The first strategy is based on ex vivo modification of cells and transplantation of the modified cells into patients. Cells that are most commonly considered therapeutic targets for LSDs are hematopoietic progenitor cells.

Substrate Reduction Therapy: SRT is based on the concept that the inhibition of specific steps of the biosynthetic pathways of substrates may reduce their flux to lysosomes and help restore the equilibrium between their synthesis and catabolism. This task is generally accomplished by using small-molecule inhibitors of enzymes involved in the biosynthesis of substrates. SRT has already been approved for clinical use for the treatment of type 1 Gaucher disease, the most prevalent LSD, due to β-glucocerebrosidase deficiency and characterized by glycosphingolipid storage, and of Niemann-Pick disease type C (NPC), a defect of intracellular cholesterol trafficking.

CRISPR Cas9: CRISPR/Cas9 (clustered regularly interspaced short palindromic repeats/caspase 9) has the potential to correct specific genetic defects without long-term deleterious side effects. CRISPR/Cas is based on the use of a guide ribonucleic acid to direct a ‘molecular scissor’ to a specific site in the genome and cut double-stranded DNA by the endonuclease caspase 9. Thereafter, the double-stranded DNA breaks are repaired by adding donor DNA, finally leading to a controlled gene modification.

In certain embodiments, methods of treating a disease or disorder associated with lysosomal dysfunction comprise treating LSDs, NCLs, idiopathic/sporadic brain disorders that include a wide range of neurological, psychiatric, or functional brain disorders. In certain embodiments, the LSDs comprise glycogen storage disease, mucopolysaccaridoses, mucolipidoses, oligosaccharidoses, lipidoses, sphingolipidoses, lysosomal transport diseases, a primary lysosomal hydrolase defect, a post-translational processing defect of lysosomal enzymes, a trafficking defect for lysosomal enzymes, a defect in lysosomal enzyme protection, a defect in soluble non-enzymatic lysosomal proteins, a transmembrane (non-enzyme) protein defect or an unclassified defect, Tay-Sachs disease, Sandhoff disease, Niemann-Pick disease, Fabry disease, Krabbe disease, Farber disease, Gaucher disease, metachromatic leukodystrophy, multiple sulphatase deficiency, mucolipidosis II, mucolipidosis III, mucopolysaccharidosis, MPS III, MPS VII, GM1 gangliosidosis, and Schindler Disease.

In certain embodiments, the NCLs (or BD) comprise CLN1 disease, CLN2 disease, CLN3 disease, CLN4 disease, CLN5 disease, CLN6 disease, CLN7 disease, CLN8 disease, CLN9 disease, CLN10 disease, CLN11 disease, CLN12 disease, CLN13 disease, and CLN14 disease.

In another embodiment, the method of treatment is directed to treating: Alzheimer's disease, Lewy body dementia (LBD), Parkinson's Disease, prion diseases, amyotrophic lateral sclerosis (ALS), frontotemporal lobar degeneration (FTLD), Pick's disease, frontotemporal dementia with parkinsonism, multiple sclerosis, Huntington's disease, multiple system atrophy (MSA), Smith Lemli Opitz Syndrome (SLOS), Tangier disease, Pelizaeus-Merzbacher Disease, progressive supranuclear palsy, spinal muscular atrophy, and several types of spinocerebellar ataxia and other ataxic conditions.

In certain embodiments, a psychiatric disorder comprises schizophrenia, mood disorders, alcoholism and other substance use disorders, autism spectrum disorder, attention deficit hyperactivity disorder, narcolepsy and other sleep disorders, a wide range of anxiety disorders.

In certain embodiments, treatment of a subject includes improving cognitive function. In certain embodiments, treatment of a subject includes treating a cognitive dysfunction, or subsets thereof. Examples of cognitive function, include, without limitation: perception, variety of types of memory including working memory, emotional regulation, awareness, attention, reasoning, thinking and capacity for judgment, flexibility of thinking, and executive functions.

In certain embodiments, treating a disease or disorder associated with lysosomal dysfunction, or one or more symptoms of a disease or disorder associated with lysosomal dysfunction includes delaying progression of the disease, or one or more symptoms of the disease over time as compared to typical disease progression. As used herein, “delaying progression of a disease or disorder associated with lysosomal dysfunction or one or more symptoms associated with a disease or disorder associated with lysosomal dysfunction over time” and the like refer to slowing and/or stopping progression of the disease or one or more symptoms of the disease (e.g., slowing and/or stopping the worsening or increasing severity of the disease or one or more symptoms of the disease) over time. Disease progression may be determined, for example, using a known scale, index, rating, or score, such as those described as examples herein, or another suitable test for assessing progression. For example, the scale, index, rating, score, or other suitable test may correspond to the progression of the disease overall or to the progression of one or more symptoms associated with the disease. In one embodiment, delaying progression of a disease or disorder associated with lysosomal dysfunction, or one or more symptoms of a disease or disorder associated with lysosomal dysfunction means that a subject's disease severity value (e.g., overall severity or severity of one or more symptoms) determined by a known scale, index, rating, score, etc., or other suitable test for evaluating severity, does not meaningfully increase (e.g., at least remains substantially constant). In one embodiment, delaying progression of a disease or disorder associated with lysosomal dysfunction, or one or more symptoms of a disease or disorder associated with lysosomal dysfunction means preventing the subject from reaching, or increasing the time taken for a subject to reach (e.g., decreasing the rate of change of increasing severity), a severity value according to a known scale, index, rating, score, etc., or other suitable test, for assessing progression compared to a value corresponding to typical disease progression. For example, progression can be said to be delayed when the time to reach a severity value takes at least 5% longer than that observed according to typical disease progression. Further for example, an increase in time of at least 10%, at least 15%, at least 20%, at least 25%, at least 30%, at least 40%, at least 50%, at least 60%, at least 70%, at least 80%, at least 90% or at least 100% is observed. The time over which the treatment delays progression of a disease or disorder associated with lysosomal dysfunction, or one or more symptoms of a disease or disorder associated with lysosomal dysfunction may coincide with the duration of treatment as described herein. In one embodiment, the treatment delays progression for at least about three months, at least about four months, at least about five months, or at least about six months. The treatment may delay progression for at least about 1 year, at least about 2 years, at least about 3 years, at least about 4 years, at least about 5 years, or at least about 10 years. The treatment may delay progression over the lifetime of the patient.

For example, to evaluate overall neurological status, mDRS, a four-domain scale (ambulation, manipulation, language and swallowing), may be applied. Cerebellar function may be evaluated using SARA, an eight-item clinical rating scale (gait, stance, sitting, speech, fine motor function and taxis; range 0-40, where 0 is the best neurological status and 40 the worst), and SCAFI, comprising the 8-m-Walking-Time (8MW; performed by having patients walking twice as quickly as possible from one line to another excluding turning), 9-Hole-Peg-Test (9HPT) and the number of “PATA” repetitions over 10 s. Subjective impairment and quality of life may be evaluated using the EQ-5D-5L questionnaire and VAS. To assess ocular motor function, 3-dimensional videooculography (EyeSeeCam) may be used to measure the peak velocity of saccades, gain of smooth pursuit, peak slow phase velocity of gaze-evoked nystagmus (gaze-holding function), peak slow phase velocity of optokinetic nystagmus, and gain of horizontal vestibulo-ocular reflex. To evaluate the cognitive state, WAIS-R or WISC-IV, and MoCA, assessing different cognitive domains, including attention and concentration, executive functions, memory, language, visuoconstructional skills, conceptual thinking, calculations, and orientation with a maximum of 30 points and a cut-off score of 26, may be used. The skilled person will know how to perform these and other such tests.

In another embodiment, is that the identification of biomarkers, expression levels, post-translational modifications, etc., can be used to determine a treatment for the disease or disorder associated with lysosomal dysfunction. Over time, the biomarkers may vary prompting the medical practitioner to adjust the subject's treatment. For example, the core CSF biomarkers (T-tau, P-tau, and A1342), CSF NFL, p-tau231 and plasma T-tau are strongly associated with Alzheimer's disease and the core biomarkers are strongly associated with mild cognitive impairment due to Alzheimer's disease.

### Candidate Therapeutic Agents

In certain embodiments, methods to identify agents (including an organic small molecule agents) with therapeutic activities that can be useful in treating a disease or disorder associated with lysosomal dysfunction are provided. In various embodiments, the screening methods of the disclosure can be performed in vitro, in cells or with transgenic animals. Preferably, a cell or animal model for a disease or disorder associated with lysosomal dysfunction, e.g. BD, SZ, AZ and other diseases are employed in the methods. The model can also be a transgenic animal displaying characteristics of a disease or disorder associated with lysosomal dysfunction. Typically, the cell or animal model is contacted or administered with a plurality of candidate agents. The cell or animal is then examined for a cellular response or phenotype evidencing potential therapeutic activities. As noted above, various therapeutic activities may be examined in these methods of the dysfunction. For example, the methods can involve testing candidate agents for ability to modulate a lysosomal enzyme. In some embodiments, the candidate agents can be screened for activity by assaying for changes in cellular autofluorescence (AF), lysosomal size, LAMP1 expression, lysosome enzymes and combinations thereof.

In some embodiments, agents identified in a cell model to possess such an activity can be further evaluated in secondary screens of animal models of, for example, BD or in clinical trials to determine activity against motor, behavioral, cognitive or other symptoms of the diseases. In some related embodiments, the disclosure also provides methods for assessing the effect of known drugs and other agents on treating a disease or disorder associated with lysosomal dysfunction. In certain embodiments, the candidate agents or potential therapeutic agents modulate several activities or functions of the lysosome in vivo or in vitro.

In certain embodiments, potential therapeutic agents identified based on the screening assays are selected for testing their therapeutic activity. In certain embodiments, the therapeutic activity of the candidate or potential therapeutic agent is monitored by cellular autofluorescence (AF), lysosomal size, LAMP1 expression, and combinations thereof.

In certain embodiments, the therapeutic activity of the candidate or potential therapeutic agent is monitored by PLA2, such as PLA2G4A or PLA2G7.

Candidate Test Agents: Various candidate agents can be employed in the screening methods of the invention, including any naturally existing or artificially generated agents. They can be of any chemistry class, such as antibodies, proteins, peptides, small organic compounds, saccharides, fatty acids, steroids, purines, pyrimidines, nucleic acids, and various structural analogs or combinations thereof. In some embodiments, the screening methods utilize combinatorial libraries of candidate agents. Combinatorial libraries can be produced for many types of compounds that can be synthesized in a step-by-step fashion. Such compounds include polypeptides, beta-turn mimetics, nucleic acids, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated herein by reference for all purposes).

Candidate agents include numerous chemical classes, though typically they are organic compounds including small organic compounds, nucleic acids including oligonucleotides, peptides or antibodies. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol. Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids; encoded peptides; random bio-oligomers); benzodiazepines (U.S. Pat. No. 5,288,514); diversomers, such as hydantoins, benzodiazepines and dipeptides; vinylogous polypeptides; nonpeptidal peptidomimetics with β-D-glucose scaffolding; analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif, 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

The screening assays of the disclosure suitably include and embody, animal models, cell-based systems and non-cell based systems. Identified genes, variants, fragments, or oligopeptides thereof are used for identifying agents of therapeutic interest, e.g. by screening libraries of compounds or otherwise identifying compounds of interest by any of a variety of drug screening or analysis techniques. The gene, allele, fragment, or oligopeptide thereof employed in such screening may be free in solution, affixed to a solid support, borne on a cell surface, or located intracellularly. The measurements will be conducted as described in detail in the examples section which follows.

In some embodiments, a method of identifying candidate therapeutic agents comprises screening a sample containing the specific target molecule in a high-throughput screening assay.

In another embodiment, a method of identifying candidate therapeutic agents for treatment of disease, comprises culturing an isolated cell expressing a target molecule, administering a candidate therapeutic agent to the cultured cell; correlating the target molecules expression, activity and/or function in the presence or absence of a candidate therapeutic agent as compared to control cells, wherein a drug is identified based on desirable therapeutic outcomes. For example, a drug which modulates levels of the target molecule, e.g. lysosomal enzyme whereby such levels are responsible for the disease state or the target molecule modulates the activity or amount of another molecule whether upstream or downstream in a pathway. In other examples the assays measure kinase activity. In other examples, the assay measure binding partners. In other examples, the assay measures amounts of candidate therapeutic agents which provide a desired therapeutic outcome.

Another suitable method for diagnosis and candidate drug discovery includes contacting a test sample with a cell expressing a target molecule, and detecting interaction of the test agent with the target molecule, an allele or fragment thereof, or expression product of the target molecule an allele or fragment thereof.

In another preferred embodiment, a sample, such as, for example, a cell or fluid from a patient is isolated and contacted with a candidate therapeutic molecule. The genes, expression products thereof, are monitored to identify which genes or expression products are regulated by the drug.

In another aspect, the disclosure provides methods for diagnosing or monitoring disease progression in subjects affected by a disease or disorder associated with lysosomal dysfunction. The therapeutic effectiveness of the agent is monitored by cellular autofluorescence (AF), lysosomal size, LAMP1 expression, and combinations thereof. These methods entail detecting and measuring in a biological sample (e.g., a tissue or body fluid sample) from the subjects the cellular autofluorescence (AF), lysosomal size, LAMP1 expression, and combinations thereof. In some methods, the biological sample is obtained from the brain of the subject. Detection and quantitation of cellular autofluorescence (AF), lysosomal size, and LAMP1 expression, in the biological sample can be readily performed in accordance with the techniques exemplified herein or protocols routinely practiced in the art.

The disclosure also provides engineered cells (e.g., neural cells) and transgenic animals expressing a lysosomal protein or enzyme. The engineered cells and transgenic animal may be used in vitro or animal models to study disease or disorder associated with lysosomal dysfunction as noted above, or to test the efficacy of therapeutic agents. The transgene is preferably present in all or substantially all of the somatic and germline cells of the transgenic animal. The polynucleotide encoding the full-length and/or mutated and/or truncated lysosomal enzyme is operably linked to one or more regulatory segments that allow the enzyme to be expressed in cells of the animal. Promoters such as the rat neuron specific enolase promoter, the prion protein promoter, human beta-actin gene promoter, human platelet derived growth factor B (PDGF-B) chain gene promoter, rat sodium channel gene promoter, mouse myelin basic protein gene promoter, human copper-zinc superoxide dismutase gene promoter, and mammalian POU-domain regulatory gene promoter can be employed in expressing the transgene. Optionally, an inducible promoter can be used. The mouse metallothionine promoter, which can be regulated by addition of heavy metals such as zinc to the mouse's water or diet, is suitable. The engineered cells or transgenic animals of the invention can be produced by the general approaches described in the art, e.g., Masliah et al, Am. J. Pathol. 148:201-10, 1996; Feany et al, Nature 404:394-8, 2000; and U.S. Pat. No. 5,811,633.

### Pharmaceutical Compositions

In certain embodiments, a pharmaceutical composition comprises at least one agent, wherein the agent modulates the function or activity of the lysosome. The pharmaceutical composition may comprise an agent e.g., troglitazone, rosuvastatin or a pharmaceutically acceptable salt thereof and a pharmaceutically acceptable carrier. Reference to the pharmaceutical composition encompasses the active agent alone (e.g., troglitazone, rosuvastatin, or a pharmaceutically acceptable salt thereof, or a statin, or Compound A) or in the form of a pharmaceutical composition.

The pharmaceutical composition may be formulated and administered to a subject in accordance with known teachings in the art. The pharmaceutical composition may take any of a number of different forms depending, in particular, on the manner in which it is to be used. Thus, for example, it may be in the form of a powder, tablet, capsule, liquid, ointment, cream, gel, hydrogel, aerosol, spray, micellar solution, transdermal patch, liposome suspension or any other suitable form that may be administered to a person or animal in need of treatment.

A “pharmaceutically acceptable carrier” as referred to herein, is any known compound or combination of known compounds that are known to those skilled in the art to be useful in formulating pharmaceutical compositions. It will be appreciated that the carrier of the pharmaceutical composition should be one which is tolerated by the subject to whom it is given.

In one embodiment, the pharmaceutically acceptable carrier may be a solid, and the composition may be in the form of a powder or tablet. A solid pharmaceutically acceptable carrier may include, but is not limited to, one or more substances which may also act as flavoring agents, buffers, lubricants, stabilizers, solubilizes, suspending agents, wetting agents, emulsifiers, dyes, fillers, glidants, compression aids, inert binders, sweeteners, preservatives, dyes, coatings, or tablet-disintegrating agents. The carrier may also be an encapsulating material. In powders, the carrier may be a finely divided solid that is in admixture with the finely divided active agents according to the invention. In tablets, the active agent may be mixed with a carrier having the necessary compression properties in suitable proportions and compacted in the shape and size desired. The powders and tablets may, for example, contain up to 99% of the active agents. Suitable solid carriers include, for example, calcium phosphate, magnesium stearate, talc, sugars, lactose, dextrin, starch, gelatin, cellulose, polyvinylpyrrolidine, low melting waxes and ion exchange resins. In another embodiment, the pharmaceutically acceptable carrier may be a gel and the composition may be in the form of a cream or the like.

The carrier may include, but is not limited to, one or more excipients or diluents. Examples of such excipients are gelatin, gum arabicum, lactose, microcrystalline cellulose, starch, sodium starch glycolate, calcium hydrogen phosphate, magnesium stearate, talcum, colloidal silicon dioxide, and the like.

In another embodiment, the pharmaceutically acceptable carrier may be a liquid. In one embodiment, the pharmaceutical composition is in the form of a solution. Liquid carriers are used in preparing solutions, suspensions, emulsions, syrups, elixirs and pressurized compositions. The agent may be dissolved or suspended in a pharmaceutically acceptable liquid carrier such as water, an organic solvent, a mixture of both or pharmaceutically acceptable oils or fats. The liquid carrier may contain other suitable pharmaceutical additives such as solubilizes, emulsifiers, buffers, preservatives, sweeteners, flavoring agents, suspending agents, thickening agents, colors, viscosity regulators, stabilizers or osmoregulators. Suitable examples of liquid carriers for oral and parenteral administration include water (partially containing additives as above, e.g. cellulose derivatives, such as sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier may also be an oily ester such as ethyl oleate and isopropyl myristate. Sterile liquid carriers are useful in sterile liquid form compositions for parenteral administration. The liquid carrier for pressurized compositions may be a halogenated hydrocarbon or other pharmaceutically acceptable propellant.

Liquid pharmaceutical compositions, which are sterile solutions or suspensions, may be utilized by, for example, intramuscular, intrathecal, epidural, intraperitoneal, intravenous and subcutaneous injection. The active agent may be prepared as a sterile solid composition that may be dissolved or suspended at the time of administration using sterile water, saline, or other appropriate sterile injectable medium.

The compositions may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compositions may also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Compositions may alternatively be administered by inhalation (e.g. intranasally). Compositions may also be formulated for topical use. For instance, creams or ointments may be applied to the skin.

The compositions may be incorporated within a slow- or delayed-release device. Such devices may, for example, be inserted on or under the skin, and the medicament may be released over weeks or even months. Such devices may be advantageous when long-term treatment is required and which may require frequent administration (e.g. at least daily administration).

In one embodiment, the pharmaceutical composition is a solid oral dosage form, such as a tablet. In tablets, the active agent may be mixed with a vehicle, such as a pharmaceutically acceptable carrier, having the necessary compression properties in suitable proportions and compacted in the shape and size desired. The tablets may contain up to 99% by weight of the active agents.

Pharmaceutical compositions in solid oral dosage form, such as tablets, may be prepared by any method known in the art of pharmacy. Pharmaceutical compositions are usually prepared by mixing the active agent with conventional pharmaceutically acceptable carriers.

A tablet may be formulated as is known in the art, for example, it can include wheat starch, pregelatinized maize (corn) starch, calcium carbonate and magnesium stearate as excipients. The same, or similar, excipients, for example, may be employed with the present disclosure.

As discussed above, the agents or the pharmaceutically acceptable salts thereof may be formulated and administered as a pharmaceutical composition taking any number of different forms. For example, the agent, or the pharmaceutically acceptable salt thereof may be formulated as a pharmaceutical composition to facilitate its delivery across the blood-brain barrier. As a further example, the agent may be formulated as a pharmaceutical composition for bypassing the blood-brain barrier. Formulations that facilitate delivery across the blood-brain barrier or that are suitable for administration in a manner that bypasses the blood-brain barrier may be used to prepare and administer the agent as described herein. Such a delivery that crosses the blood-brain barrier or that is suitable for administration in a manner that bypasses the blood-brain barrier can be deemed “direct delivery” as used herein.

In one embodiment, the pharmaceutical composition (e.g., comprising troglitazone, rosuvastatin or the combination thereof) is formulated for nanodelivery, e.g., colloidal drug-carrier systems. Suitable examples include but are not limited to liposomes, nanoparticles (e.g., polymeric, lipid and inorganic nanoparticles), nanogels, dendrimers, micelles, nanoemulsions, polymersomes, exosomes, and quantum dots. See, e.g., Patel et al., “Crossing the Blood-Brain Barrier: Recent Advances in Drug Delivery to the Brain,” CNS Drugs 31:109-133 (2017); Kabanov et al., “New Technologies for Drug Delivery across the Blood Brain Barrier,” Curr Pharm Des., 10(12):1355-1363 (2004); Cheng et al., “Highly Stabilized Curcumin Nanoparticles Tested in an In Vitro Blood-Brain Barrier Model and in Alzheimer's Disease Tg2576 Mice,” The AAPS Journal, vol. 15, no. 2, pp. 324-336 (2013); Lahde et al. “Production of L-Leucine Nanoparticles under Various Conditions Using an Aerosol Flow Reactor Method,” Journal of Nanomaterials, vol. 2008, article ID 680897 (2008). For instance, as used herein, direct delivery may encompass formulations for nanodelivery.

In one embodiment, the pharmaceutical composition (e.g., comprising troglitazone, rosuvastatin or the combination thereof, or a statin, or Compound A) is formulated for direct delivery to the central nervous system (CNS), such as by injection or infusion. Formulations for and methods of direct delivery to the CNS are known in the art. See, e.g., U.S. Pat. No. 9,283,181. Examples of such administration include but are not limited to intranasal, intraventricular, intrathecal, intracranial, administration via nasal mucosal grafting, intravenous administration (e.g. IV injection) and oral administration (P.O).

In one embodiment, the pharmaceutical composition is formulated for (and administered by) intranasal delivery. See, e.g., Hanson et al., BMC Neurosci. 9(Suppl 3):S5 (2008). In one embodiment, the pharmaceutical composition is formulated for (and administered by) delivery via a nasal mucosal graft. In one embodiment, the pharmaceutical composition is formulated for (and administered by) intracerebroventricular injection or infusion. In another embodiment, the pharmaceutical composition is formulated for (and administered by) intrathecal intracisternal injection or infusion. In one embodiment, the pharmaceutical composition is formulated for (and administered by) intrathecal lumbar injection or infusion. For example, the active agent may be formulated for intrathecal administration and/or administered intrathecally in the same or a similar manner discussed by Ory et al., “Intrathecal 2-hydroxypropyl-.beta.-cyclodextrin decreases neurological disease progression in Niemann-Pick disease, type C1: a non-randomized, open-label, phase 1-2 trial,” Vol. 390, Issue 10104, pp. 1758-1768 (2017).

Various techniques may be used including, without limitation, injection through a burrhole or cisternal or lumbar puncture or the like as known in the art. Various devices, whether internal (e.g., implanted) or external, may be used for delivery as known in the art, such as pumps, catheters, reservoirs, etc. In one embodiment, the administration interval is once every two weeks.

In yet a further embodiment, the pharmaceutical composition is formulated for (and administered by) intravenous injection or infusion. For example, troglitazone, rosuvastatin or the combination thereof, may be formulated for intravenous administration using a needle or tube. In another embodiment, the pharmaceutical composition is formulated for (and administered by) oral administration.

In one embodiment, the administration interval is once every month. In one embodiment, the administration interval is once every two months. In one embodiment, the administration interval is twice per month. In one embodiment, the administration interval is once every week. In one embodiment, the administration interval is twice or several times per week. In one embodiment, the administration interval is daily. In one embodiment, the administration is continuous, such as continuous infusion.

The following non-limiting examples are illustrative.

### EXAMPLES

Without wishing to be bound by theory, it was hypothesized that lysosomal deficit can be a primary driver for neuronal dysfunction.

### Example 1

### Methods

Recruitment of study participants: Schizophrenia (SZ) patients and age-, gender-, and race-matched controls were recruited in the Johns Hopkins Schizophrenia Center.

Neurocognitive tests: Cognitive test battery covering 6 cognitive domains (cognitive flexibility, attention, processing speed, verbal learning/memory, visual learning/memory, and fluency) were performed by using our published protocol (Horiuchi et al., Transl Psychiatry, 2016). Cognitive flexibility was evaluated via Wisconsin Card Sorting Test (WCST).

Electron microscopy for human cells: 70 nm sections of human blood cells were viewed on a Hitachi H 7600 TEM operating at 80 kV, and digital images were captured with an ER-50 AMT (5 megapixel) CCD camera. Grids with no first antibodies were served as negative controls. All the data was obtained through the core facility service at Johns Hopkins.

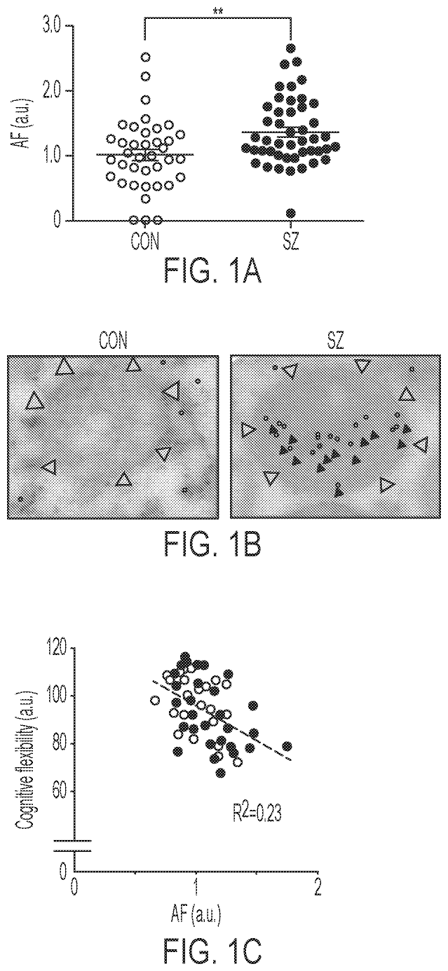

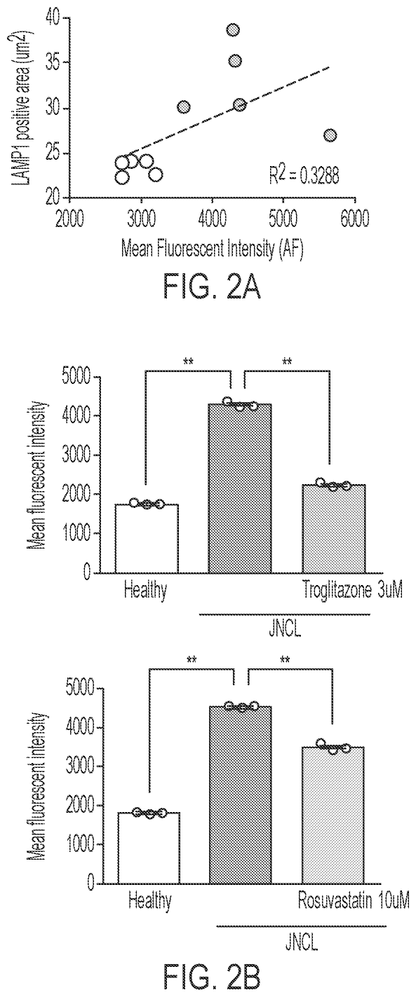

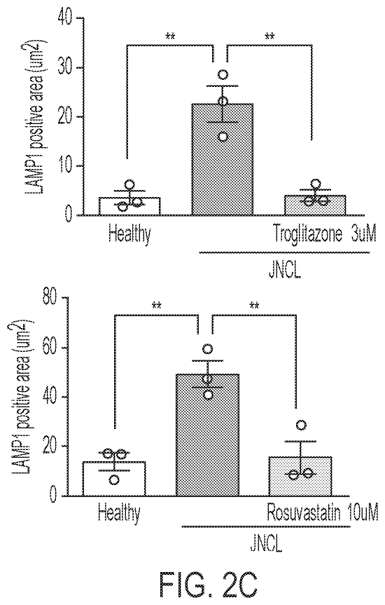

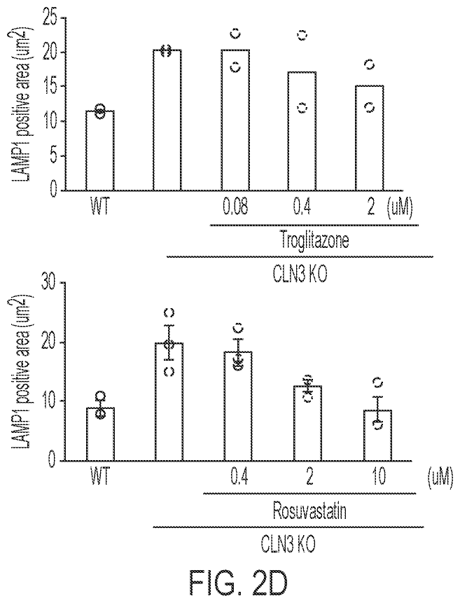

Autofluorescence (AF): AF levels in human blood cells were measured in 30,000 cells using flow cytometry at FL-1 with the BD FACSCalibur™ (BD Biosciences) (Excitation; 488 nm, Emission; 530±15 nm) (for FIGS. 1A-1C). AF levels in lymphoblasts were measured in 20,000 cells using flow cytometry with Attune Flow Cytometer (Thermo Fisher Scientific) (Excitation; 488 nm, Emission; 530 nm) (for FIGS. 2A and 2B).

LAMP1 staining: Lymphoblasts were fixed with methanol and incubated overnight at 4° C. with anti-human LAMP1 antibody (1:400, Cell Signaling Technology) followed by secondary antibody (Alexa Fluor 488 donkey anti-rabbit IgG, 1:1000, Thermo Fisher Scientific). Neuronal cells differentiated from iPS cells were fixed with 4% paraformaldehyde (PFA) and incubated overnight at 4° C. with anti-human LAMP1 antibody (1:400, Cell Signaling Technology) followed by a secondary antibody (Alexa Fluor 488 donkey anti-rabbit IgG, 1:1000, Thermo Fisher Scientific). White blood cells from mice were fixed with methanol and incubated overnight at 4° C. with LAMP1 antibody (1:1000, Abcam) followed by a secondary antibody (Alexa Fluor 488 donkey anti-rabbit IgG, 1:1000, Thermo Fisher Scientific). KEYENCE BZ-X710 and BX-Z analyzer software were used for imaging quantification.

Novel Object Recognition Test (NORT): The test was carried out according to the previously validated method (Hatayama et al., Neuroscience 265 (2014) 217-25). All mice were habituated for 1 hour to the NORT arena one day before the test day. On the day of testing, the mice were given two 5-min trials (T1 with two identical objects and T2 with a familiar object from T1 and a novel object), separated by 1-hour interval. The discrimination index (DI) ((time spent exploring the novel object−time spent exploring the familiar object)/total exploration time) was calculated for T2 trials.

Histological analysis: Mice were sacrificed and brains were dissected and fixation was continued overnight with 4% paraformaldehyde. Brain sections were incubated overnight at 4° C. with antibodies specific for mouse LAMP1 (1:200, Abcam) followed by a secondary antibody (Histofine Simple Stain Mouse MAX-PO (R), Nichirei Bioscience). For imaging quantification, individual slice was analyzed using Aperio AT-2 and ImageScope (Leica).

Isolation of white blood cells from peripheral blood of mice: 20 μL of blood of mice was lysed with ACK lysing buffer (Lonza) for 1 min. After washing, cells were resuspended with RPMI.

RNA-sequencing in iPS cell-derived neurons: Cells were differentiated from iPS cells and treated either with 0.1% DMSO or Compound A for 2 weeks. RNAs were extracted using RNeasy Mini Kit (QIAGEN). The libraries for RNA-sequencing were constructed by SMART-Seq v4 Ultra Low Input RNA Kit (Takara bio).

### Results

The Johns Hopkins Schizophrenia Center has recruited patients with SZ for the past decade, with extensive tissue collection from the patients and matched controls. One of the aims of this effort was to establish good biomarkers that are associated with the disease and specific clinical/behavioral manifestations. During such an effort, an interesting finding was the discovery that cellular autofluorescence (AF) is elevated in peripheral blood cells from SZ patients compared with those from healthy controls (FIG. 1A). Elevated AF has been frequently reported in BD, so it was hypothesized that the lysosomal change could be observed in blood cells of SZ patients. The cells were examined at the electron microscopy level, and found aberrantly enlarged lysosomes (a sign of lysosomal functional deficits) in cells from some patients with SZ (FIG. 1B). SZ includes many clinical manifestations, it was examined which of these specific clinical dimensions are AF-related. Interestingly, AF is negatively correlated with cognitive function, but not with positive and negative symptoms (FIG. 1C). A systematic assessment of multiple cognitive subdimensions elucidated that cognitive flexibility is a specific dimension that showed correlation with AF (FIG. 1C).

BD cell and animal models, in particular Cln3 genetic models were studied next. These genetic models recapitulate lysosomal deficits relevant to the pathophysiology of BD. The lysosomal deficits in lymphoblasts from BD patients with a Cln3 mutation were confirmed by using two independent measurements: LAMP1 expression and AF levels. Correlations between LAMP1 and AF (FIG. 2A) were observed in this study. Indeed, the lysosomal deficits were confirmed with these two measurements in a consistent manner (FIGS. 2B, 2C). Encouraged by confirmation of lysosomal deficit in BD patient cells, it was next addressed whether the disease was treatable. To look for a promising compound for treatment in an efficient manner, a hypothesis-driven approach was taken in which chemicals known to affect glycolipid metabolism were hypothesized that they may directly influence lysosomal functions. Accordingly, about 50 chemicals were tested in this category. It was found that troglitazone and rosuvastatin could ameliorate the lysosomal deficits found in BD lymphoblasts (FIGS. 2C, 2D). Discovery of these beneficial impacts is novel, and nobody has suggested or predicted. Finally, an induced pluripotent stem (iPS) cell line was examined, in which a BD-relevant Cln3 mutation was introduced. The iPS cells were differentiated into neurons and confirmed the BD-relevant lysosomal deficits (FIG. 2D). Consistent with the observations in BD lymphoblasts, the treatment effects of troglitazone were also detected in the iPS cell-derived neurons with the Cln3 mutation (FIG. 2D).

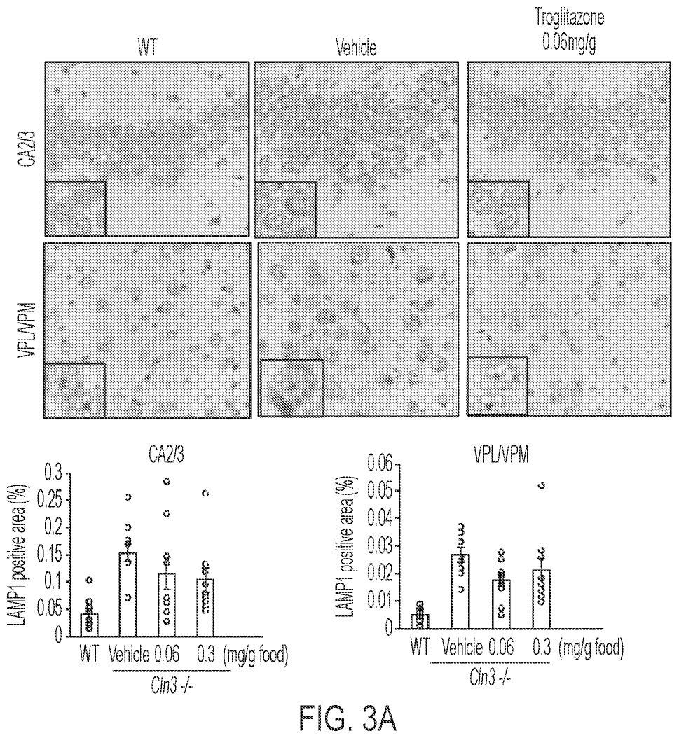

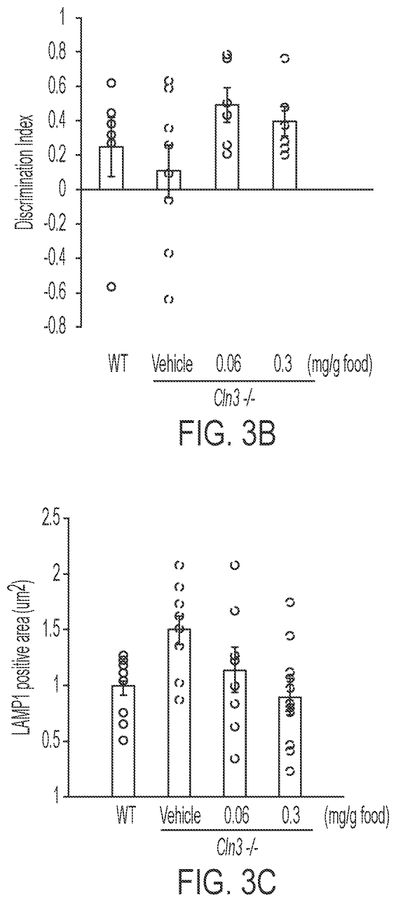

In postmortem brains of patients with BD, there are several specific brain regions in which pathology is more prominent: the hippocampus and thalamus. Accordingly, the potential pathology was examined in these regions and the sign of lysosomal deficits were observed in tissue sections of the hippocampus and thalamus in one of the most representative BD animal models (Cln3 knockout mice) (FIG. 3A). Of the most importance, consistent with the novel finding in BD cell models, administration of troglitazone ameliorated the pathology (FIG. 3A). As described in the introduction section, patients with BD display multiple types of neurological and psychiatric manifestations. Several efforts of addressing neurological manifestations, such as seizure, have been made and shown some good outcome in patient care of BD. However, there is no way of addressing cognitive deficit in BD. Therefore, this was addressed through the animal models. First observed, were cognitive deficits in the BD animal models in novel object recognition test (FIG. 3B). Thus, it was tested whether troglitazone could ameliorate the deficits, and indeed observed the beneficial outcome (FIG. 3B). Even at the level of animal models, this is the first demonstration of BD-relevant cognitive deficit and its amelioration by a small compound. In a clinical setting, peripheral biomarkers are always very important for the evaluation of the disease pathology and treatment response. Accordingly, the next question is whether or not the lysosomal deficit relevant to BD can be seen in peripheral cells of animal models. The lysosomal deficit was tested in peripheral blood cells and deficit was observed. Even more importantly, administration of troglitazone in vivo resulted in the amelioration of the blood pathology, and such treatment effects on blood cells were consistent with those for neurons and for behavioral changes (FIG. 3C).

### SUMMARY

Through the experiments herein, the inventors have obtained promising evidence that SZ cells also have deficits associated with lysosomes. These include: an elevated AF which is similar to BD, the deficit found in SZ shows correlation with dysfunction in a specific cognitive dimension, and BD animal model also shows lysosome deficit that seems to underlie cognitive changes. Taken together, it is now logically conceivable that both rare genetic diseases (e.g., BD) and idiopathic/sporadic brain disorders (e.g, SZ, AD) may share common lysosomal abnormalities underlining functional deficits. Further experiments will solidify this novel concept. The experimental data also indicate the potential and likelihood that a peripheral biomarker is established and associated with lysosomal deficit, paying attention to AF. Lastly, the result also shows that lysosomal deficit and resultant outcomes, including behavioral changes, are treatable by small compounds.

### Example 2: Synthesis of Compound A (8-{[2-(dimethylamino)ethyl]amino}-1-(2-methylpropyl)-1,2,3,4-tetrahydro-6H-pyrimido[2,1-b]quinazolin-6-one)

Compound A can be synthesized as demonstrated in the following Scheme 1.

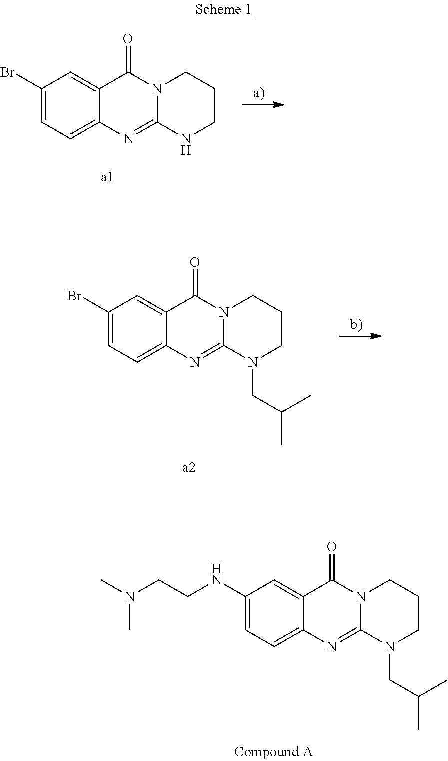 [SMILES: O=c1c2cc(Br)ccc2nc2n1CCCN2 ; CC(C)CN1CCCn2c1nc1ccc(Br)cc1c2=O ; CC(C)CN1CCCn2c1nc1ccc(NCCN(C)C)cc1c2=O]

8-bromo-1,2,3,4-tetrahydro-6H-pyrimido[2,1-b]quinazolin-6-one (compound a1) is commercially available product and also may be synthesized according to a method described in Tetrahedron (2007), 63(4), 847-854 or other method.

Synthesis of 8-bromo-1-(2-methylpropyl)-1,2,3,4-tetrahydro-6H-pyrimido[2,1-b]quinazolin-6-one (Compound a2): To a mixture of Compound a1 (5 g) and cesium carbonate (11.6 g) in DMF (50 ml) was added dropwise 1-bromo-2-methylpropane (3.1 ml). After stirring for 5 hours at a temperature of 100° C., the reaction mixture was quenched with water (300 ml). The resulting solid was collected by filtration and washed with water and hexane. The resulting white solid was dried under reduced pressure at a temperature of 50° C. to give Compound a1 (5.1 g). 1H-NMR (400 MHz, CDCl₃) δ: 8.15 (1H, d, J=2.4 Hz), 7.55 (1H, d, J=2.4 Hz), 7.53 (1H, d, J=1.8 Hz), 7.13 (1H, d, J=8.5 Hz), 4.06 (2H, dd, J=6.1, 6.1 Hz), 3.49 (2H, d, J=7.9 Hz), 3.44 (2H, t, J=5.8 Hz), 2.23-2.13 (1H, m), 2.11-2.04 (2H, m), 0.93 (6H, d, J=6.7 Hz).

Synthesis of 8-{[2-(dimethylamino)ethyl]amino}-1-(2-methylpropyl)-1,2,3,4-tetrahydro-6H-pyrimido[2,1-b]quinazolin-6-one (Compound A): 4-methyltetrahydropyran (15 ml) solution of Compound a2 (450 mg), N,N-dimethylethane-1,2-diamine (139 μl), Brettphos Pd G3 (116 mg) and sodium tert-butoxide (184 mg) was stirred for 3 hours at a temperature of 100° C. Water (50 ml) was added to the reaction mixture followed by extraction with ethyl acetate. The combined organic layers were dried MgSO₄, filtered and concentrated. The crude product was added to amino silica gel column and was eluted with Hexane/AcOEt then AcOEt/MeOH to give title compound (235 mg) as a yellow solid. 1H-NMR (400 MHz, CD3OD) δ: 7.90 (1H, s), 7.14 (1H, d, J=8.5 Hz), 7.10-7.03 (2H, m), 4.07 (2H, dd, J=5.8, 5.8 Hz), 3.50-3.45 (4H, m), 3.26 (2H, t, J=6.7 Hz), 2.62 (2H, t, J=6.7 Hz), 2.27-2.15 (1H, m), 2.13-2.05 (2H, m), 0.95 (6H, d, J=6.7 Hz).

### Example 3

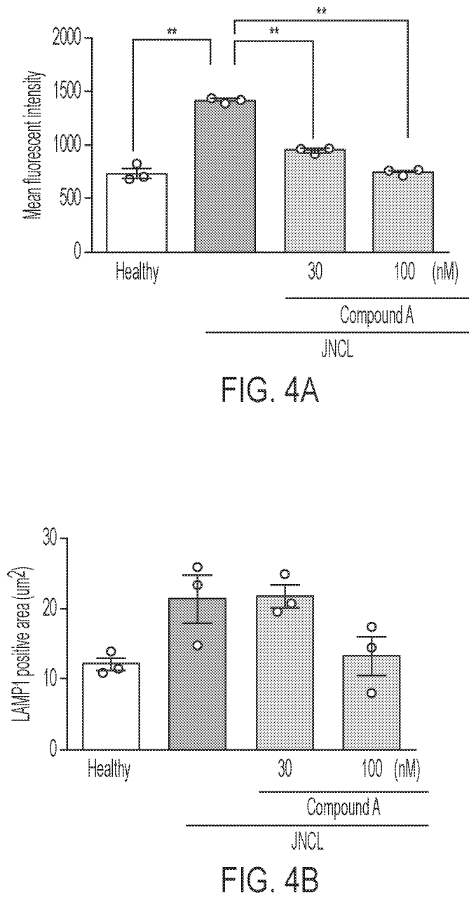

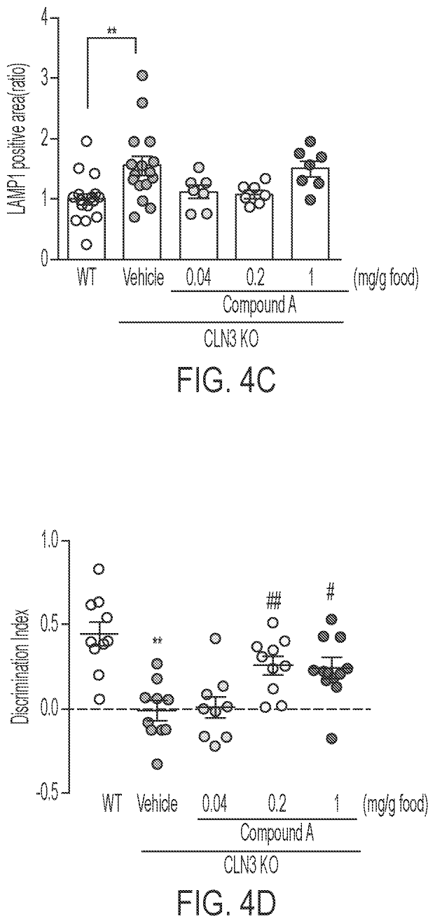

FIGS. 4A to 4D show beneficial effects of Compound A on Batten-associated pathology in human cells, and mouse disease model (cell pathology, behavior). FIG. 4A shows amelioration of elevated autofluorescence (AF) in Batten disease (BD) lymphoblasts by Compound A. FIG. 4 B shows amelioration of elevated LAMP1 signal in Batten disease (BD) lymphoblasts by Compound A. FIG. 4C shows amelioration of elevated LAMP1 signal in peripheral white blood cells of BD mice [CLN3 knockout (KO) mice] by Compound A. FIG. 4D shows amelioration of cognitive deficits (novel object recognition test) of BD mice by Compound A.

### Example 4

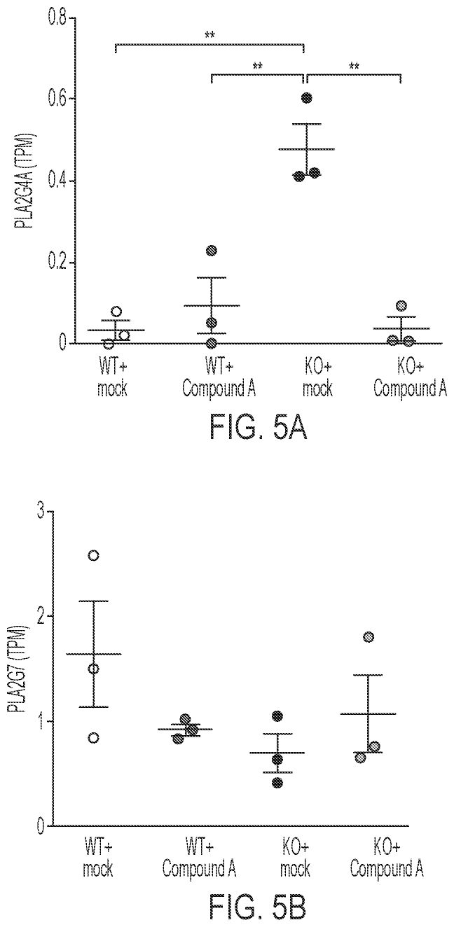

FIGS. 5A and 5B show expression control of PLA2G4A (FIG. 5A) and PLA2G7 (FIG. 5B) by Compound A. Compound A significantly influenced the expression of PLA2 genes, which may underlie a main mechanism of pathological amelioration of Batten CLN3 disease. The data was obtained through RNA-sequencing analysis in which we compared gene expression among the groups (i) wild-type (WT) neurons+mock, (ii) WT neurons+Compound A, (iii) Batten cell model CLN3 knockout (KO) neurons+mock, and (iv) KO neurons+Compound A. Neurons were prepared by differentiating human induced pluripotent stem cells.

### Example 5

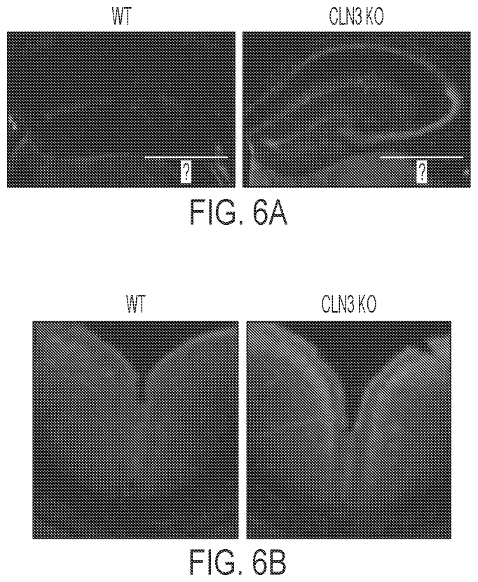

FIGS. 6A and 6B show connection between the cellular/tissue pathology with the behavioral/neuropsychiatric phenotypes by conducting the histological analysis of autofluorescence (AF). FIG. 6A shows that the Batten CLN3 disease pathology in the brain was emphasized in the hippocampal CA3 region, and FIG. 6B also shows that the cerebral cortex was involved in CLN3 KO mice. Thus, the influence of Compound A on PLA2 genes may be a main mechanism of pathological amelioration, partly because PLA2 genes are directory involved in the metabolism of G3PI.

### Other Embodiments

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of treating a subject at risk of, or diagnosed with a disease or disorder associated with lysosomal dysfunction, comprising: administering to the subject a therapeutically effective dose of one or more agents, wherein the one or more agents modulate the function or activity of a lysosome, thereby treating a subject at risk of, or diagnosed with a disease or disorder associated with lysosomal dysfunction.

2. The method of claim 1, wherein the function or activity of the lysosome is determined by modulation of cellular autofluorescence (AF), lysosomal size, LAMP1 expression, lysosome enzymes or combinations thereof.

3. (canceled)

4. A method of treating a subject suffering from or susceptible a lysosomal storage disorder (LSD) or a neuronal ceroid lipofuscinose (NCL), comprising: administering to the subject an effective amount of one or more agents that modulate the function or activity of a lysosome.

5. The method of claim 1 wherein the one or more agents comprise one or more organic small molecule compounds.

6. A method of treating a subject suffering from or susceptible to a lysosomal storage disorder (LSD) or a neuronal ceroid lipofuscinose (NCL), comprising: administering to the subject an effective amount of one or more agents selected from: i) troglitazone and/or rosuvastatin; ii) one or more activators of peroxisome proliferator-activated receptors (PPARs); iii) one or more thiazolidinedione compounds; iv) one or more statin compounds; and/or v) Compound A.

7. The method of claim 4 wherein the one or more agents comprise troglitazone and/or rosuvastatin.

8. The method of claim 4 wherein the agent comprises one or more small molecule compounds, antisense oligonucleotides, siRNA reagents, antibodies, antibody fragments, single chain antibodies, antibody mimetics, peptoids, aptamers; enzymes, peptides, organic or inorganic molecules, natural or synthetic compounds or combinations thereof.

9. The method of claim 4 wherein the one or more agents are small molecules that modulate glycolipid metabolism.

10. The method of claim 4 wherein the one or more agent comprises one or more activators of peroxisome proliferator-activated receptors (PPARs).

11. The method of claim 4 wherein the one or more agents comprise one or more activators of PPAR-y.

12. The method of claim 4 wherein the one or more agents comprise one or more thiazolidinedione compounds.

13. The method of claim 4 wherein the one or more agents comprises pioglitazone and/or rosiglitazone.

14. The method of claim 4 wherein the one or more agents comprise one or more statin compounds.

15. The method of claim 4 wherein the one or more agents comprise one or more of atorvastatin, lovastatin, pitavastatin, pravastatin, rosuvastatin and/or simvastatin.

16. The method of claim 1 wherein the disease or disorder associated with lysosomal dysfunction comprises lysosomal storage disorders (LSDs), neuronal ceroid lipofuscinoses (NCLs), idiopathic/sporadic brain disorders that include neurological, psychiatric, or functional brain disorders.

17. The method of claim 16 wherein LSDs comprise glycogen storage disease, mucopolysaccaridoses, mucolipidoses, oligosaccharidoses, lipidoses, sphingolipidoses, lysosomal transport diseases, a primary lysosomal hydrolase defect, a post-translational processing defect of lysosomal enzymes, a trafficking defect for lysosomal enzymes, a defect in lysosomal enzyme protection, a defect in soluble non-enzymatic lysosomal proteins, a transmembrane (non-enzyme) protein defect or an unclassified defect, Tay-Sachs disease, Sandhoff disease, Niemann-Pick disease, Fabry disease, Krabbe disease, Farber disease, Gaucher disease, metachromatic leukodystrophy, multiple sulphatase deficiency, mucolipidosis II, mucolipidosis III, mucopolysaccharidosis, MPS III, MPS VII, GM1 gangliosidosis, and Schindler Disease.

18. The method of claim 16, wherein the NCLs (or BD) comprise CLN1 disease, CLN2 disease, CLN3 disease, CLN4 disease, CLN5 disease, CLN6 disease, CLN7 disease, CLN8 disease, CLN9 disease, CLN10 disease, CLN11 disease, CLN12 disease, CLN13 disease or CLN14 disease.

19. The method of claim 1 wherein the subject is suffering from a neurological disease that is: Alzheimer's disease, Lewy body dementia (LBD), Parkinson's Disease, prion diseases, amyotrophic lateral sclerosis (ALS), frontotemporal lobar degeneration (FTLD), Pick's disease, frontotemporal dementia with parkinsonism, multiple sclerosis, Huntington's disease, multiple system atrophy (MSA), Smith Lemli Opitz Syndrome (SLOS), Tangier disease, Pelizaeus-Merzbacher Disease, progressive supranuclear palsy, spinal muscular atrophy, or spinocerebellar ataxia and other ataxic conditions.

20. The method of claim 1 wherein the subject is suffering from a psychiatric disorder that is schizophrenia, mood disorders, alcoholism and other substance use disorders, autism spectrum disorder, attention deficit hyperactivity disorder, narcolepsy or other sleep disorders, or an anxiety disorder.

21. The method of claim 4 further comprising administering a secondary therapeutic agent distinct from the one or more agents.

22-49. (canceled)

50. A method of treating a subject at risk of, or diagnosed with Batten disease, comprising: administering to the subject a therapeutically effective amount of one or more activators of peroxisome proliferator-activated receptors (PPARs), one or more thiazolidinedione compounds, and/or one or more statin compounds.

51-53. (canceled)

54. A method of treating a subject at risk of, or diagnosed with Batten disease, comprising: administering to the subject a therapeutically effective amount of one or more small molecule compounds that can modulate the function or activity of a lysosome.

55-58. (canceled)

59. The method of claim 4 wherein the one or more agents comprise one or more organic small molecule compounds.
